SEQUENCE LISTING (232 sequences)

SEQ ID NO: 1 - protein, 130 aa, Artificial Sequence
  Synthetic
  MISC_FEATURE (1)..(1): optionally absent
  MISC_FEATURE (2)..(2): Xaa is S, A, D, G, L, or is optionally absent
  MISC_FEATURE (3)..(3): Xaa is H, Q, R, P, or K
  MISC_FEATURE (4)..(4): Xaa is S, T, R, N, or I
  MISC_FEATURE (5)..(5): Xaa is S, A, G, or V
  MISC_FEATURE (6)..(6): Xaa is H, Q, K, or E
  MISC_FEATURE (7)..(7): Xaa is G, V, or E
  MISC_FEATURE (8)..(8): Xaa is A, T, P or V
  MISC_FEATURE (9)..(9): Xaa is I or V
  MISC_FEATURE (10)..(10): Xaa is K or E
  MISC_FEATURE (11)..(11): Xaa is S, A or V
  MISC_FEATURE (12)..(12): Xaa is T, A or V
  MISC_FEATURE (15)..(15): Xaa is D or E
  MISC_FEATURE (16)..(16): Xaa is S, F, Y or L
  MISC_FEATURE (17)..(17): Xaa is A, V or L
  MISC_FEATURE (19)..(19): Xaa is S, A, V or G
  MISC_FEATURE (20)..(20): Xaa is F, C or Y
  MISC_FEATURE (21)..(21): Xaa is N or K
  MISC_FEATURE (22)..(22): Xaa is S, N, C, R, P or G
  MISC_FEATURE (23)..(23): Xaa is M, N or K
  MISC_FEATURE (24)..(24): Xaa is  N or D
  MISC_FEATURE (25)..(25): Xaa is A, T, G or V
  MISC_FEATURE (26)..(26): Xaa is A or T
  MISC_FEATURE (27)..(27): Xaa is D or G
  MISC_FEATURE (28)..(28): Xaa is L or V
  MISC_FEATURE (29)..(29): Xaa is A or V
  MISC_FEATURE (30)..(30): Xaa is S, C, N, R or G
  MISC_FEATURE (31)..(31): Xaa is N or K
  MISC_FEATURE (32)..(32): Xaa is S or Y
  MISC_FEATURE (33)..(33): Xaa is T, M, K, I, L or V
  MISC_FEATURE (34)..(34): Xaa is N or D
  MISC_FEATURE (35)..(35): Xaa is D or G
  MISC_FEATURE (36)..(36): Xaa is A, P or V
  MISC_FEATURE (37)..(37): Xaa is S, A, T, P, E or V
  MISC_FEATURE (39)..(39): Xaa is F or Y
  MISC_FEATURE (40)..(40): Xaa is P or L
  MISC_FEATURE (41)..(41): Xaa is Q, M, P or L
  MISC_FEATURE (42)..(42): Xaa is D, G or E
  MISC_FEATURE (44)..(44): Xaa is A, T,  or V
  MISC_FEATURE (45)..(45): Xaa is H, S, P, R,  or L
  MISC_FEATURE (46)..(46): Xaa is A  or V
  MISC_FEATURE (47)..(47): Xaa is D, G or V
  MISC_FEATURE (49)..(49): Xaa is C, P or R
  MISC_FEATURE (50)..(50): Xaa is Q or R
  MISC_FEATURE (51)..(51): Xaa is D, N or Y
  MISC_FEATURE (52)..(52): Xaa is S, T or I
  MISC_FEATURE (53)..(53): Xaa is Q, P, E or L
  MISC_FEATURE (54)..(54): Xaa is R, K or E
  MISC_FEATURE (55)..(55): Xaa is M or L
  MISC_FEATURE (56)..(56): Xaa is W or L
  MISC_FEATURE (57)..(57): Xaa is Q or L
  MISC_FEATURE (58)..(58): Xaa is D or G
  MISC_FEATURE (59)..(59): Xaa is Q or L
  MISC_FEATURE (60)..(60): Xaa is T, M, K, I or L
  MISC_FEATURE (62)..(62): Xaa is T, M,  or L
  MISC_FEATURE (63)..(63): Xaa is C  or G
  MISC_FEATURE (64)..(64): Xaa is M or V
  MISC_FEATURE (65)..(65): Xaa is S, N or C
  MISC_FEATURE (66)..(66): Xaa is D, G or E
  MISC_FEATURE (67)..(67): Xaa is P, L or V
  MISC_FEATURE (68)..(68): Xaa is K or E
  MISC_FEATURE (69)..(69): Xaa is S, F, or L
  MISC_FEATURE (71)..(71): Xaa is S, A, T, P,  or I
  MISC_FEATURE (72)..(72): Xaa is Q, M,  or L
  MISC_FEATURE (73)..(73): Xaa is N, D, G or V
  MISC_FEATURE (75)..(75): Xaa is Q or R
  MISC_FEATURE (76)..(76): Xaa is K, G or E
  MISC_FEATURE (77)..(77): Xaa is S, C or N
  MISC_FEATURE (79)..(79): Xaa is D, Y or V
  MISC_FEATURE (80)..(80): Xaa is F, I or V
  MISC_FEATURE (81)..(81): Xaa is A, T, P or V
  MISC_FEATURE (82)..(82): Xaa is S, F or Y
  MISC_FEATURE (83)..(83): Xaa is E or V
  MISC_FEATURE (84)..(84): Xaa is S or G
  MISC_FEATURE (86)..(86): Xaa is S, N, I, R or G
  MISC_FEATURE (87)..(87): Xaa is F, I, or L
  MISC_FEATURE (88)..(88): Xaa is S, C, or R
  MISC_FEATURE (89)..(89): Xaa is A, P, L or V
  MISC_FEATURE (90)..(90): Xaa is R or K
  MISC_FEATURE (91)..(91): Xaa is S or G
  MISC_FEATURE (92)..(92): Xaa is S or P
  MISC_FEATURE (93)..(93): Xaa is S, D, G or V
  MISC_FEATURE (94)..(94): Xaa is Q, T, N, R, P, or K
  MISC_FEATURE (96)..(96): Xaa is S, C, N, I or G
  MISC_FEATURE (97)..(97): Xaa is R, K or E
  MISC_FEATURE (98)..(98): Xaa is R, M or L
  MISC_FEATURE (99)..(99): Xaa is A or V
  MISC_FEATURE (100)..(100): Xaa is D or G
  MISC_FEATURE (101)..(101): Xaa is M, N, I or V
  MISC_FEATURE (102)..(102): Xaa is A, T or V
  MISC_FEATURE (103)..(103): Xaa is C or G
  MISC_FEATURE (104)..(104): Xaa is N, I, K or E
  MISC_FEATURE (105)..(105): Xaa is F or Y
  MISC_FEATURE (106)..(106): Xaa is E or V
  MISC_FEATURE (107)..(107): Xaa is M, K, G, E or V
  MISC_FEATURE (110)..(110): Xaa is R or G
  MISC_FEATURE (111)..(111): Xaa is N or K
  MISC_FEATURE (112)..(112): Xaa is A or G
  MISC_FEATURE (113)..(113): Xaa is Q, D, P, R, K or L
  MISC_FEATURE (114)..(114): Xaa is N, D, P, G or Y
  MISC_FEATURE (115)..(115): Xaa is P or G
  MISC_FEATURE (116)..(116): Xaa is S, D, P or G
  MISC_FEATURE (117)..(117): Xaa is S, W, R or L
  MISC_FEATURE (118)..(118): Xaa is S, T or K
  MISC_FEATURE (119)..(119): Xaa is F or L
  MISC_FEATURE (121)..(121): Xaa is H, C, R,  or G
  MISC_FEATURE (122)..(122): Xaa is S, A, T, I  or V
  MISC_FEATURE (123)..(123): Xaa is T, I  or R
  MISC_FEATURE (124)..(124): Xaa is F, A, S, T  or V
  MISC_FEATURE (125)..(125): Xaa is S  or N
  MISC_FEATURE (126)..(126): Xaa is Q, M, P or L
  MISC_FEATURE (127)..(127): Xaa is N, D, G, E, or V
  MISC_FEATURE (128)..(128): Xaa is T, P or L
  MISC_FEATURE (129)..(129): Xaa is S, A, T, Y, V or optionally absent
  MISC_FEATURE (130)..(130): Xaa is N, R, K, E or optionally absent
MXXXXXXXXXXXLAXXXKXXXXXXXXXXXXXXXXXXXIXXXXMXXXXGXXXXXXXXXXXXDXXXXXXXXTXXXVXXXGXXXXXXGXXXXXXXXXDXXXXXXXXXXXXVWXXXXXXXXXXYXXXXXXXXXX

SEQ ID NO: 2 - protein, 130 aa, Artificial Sequence
  Synthetic
  MISC_FEATURE (1)..(1): optionally absent
  MISC_FEATURE (2)..(2): Xaa is D, G ,L or is optionally absent
  MISC_FEATURE (3)..(3): Xaa is Q or P
  MISC_FEATURE (4)..(4): Xaa is S or T
  MISC_FEATURE (6)..(6): Xaa is H or K
  MISC_FEATURE (17)..(17): Xaa is L or V
  MISC_FEATURE (19)..(19): Xaa is A or V
  MISC_FEATURE (20)..(20): Xaa is F or Y
  MISC_FEATURE (22)..(22): Xaa is S or G
  MISC_FEATURE (27)..(27): Xaa is D or G
  MISC_FEATURE (35)..(35): Xaa is D or G
  MISC_FEATURE (37)..(37): Xaa is A or V
  MISC_FEATURE (45)..(45): Xaa is R or P
  MISC_FEATURE (51)..(51): Xaa is N or Y
  MISC_FEATURE (52)..(52): Xaa is S or I
  MISC_FEATURE (54)..(54): Xaa is R or K
  MISC_FEATURE (60)..(60): Xaa is M or I
  MISC_FEATURE (62)..(62): Xaa is M or T
  MISC_FEATURE (66)..(66): Xaa is G or E
  MISC_FEATURE (67)..(67): Xaa is P or L
  MISC_FEATURE (69)..(69): Xaa is F or L
  MISC_FEATURE (71)..(71): Xaa is T or I
  MISC_FEATURE (72)..(72): Xaa is M or L
  MISC_FEATURE (73)..(73): Xaa is D, N or V
  MISC_FEATURE (76)..(76): Xaa is K or E
  MISC_FEATURE (82)..(82): Xaa is F or Y
  MISC_FEATURE (86)..(86): Xaa is S or R
  MISC_FEATURE (93)..(93): Xaa is D or G
  MISC_FEATURE (94)..(94): Xaa is P or K
  MISC_FEATURE (100)..(100): Xaa is D or G
  MISC_FEATURE (104)..(104): Xaa is I or K
  MISC_FEATURE (114)..(114): Xaa is D or G
  MISC_FEATURE (121)..(121): Xaa is H or R
  MISC_FEATURE (127)..(127): Xaa is D or G
  MISC_FEATURE (129)..(129): optionally absent
  MISC_FEATURE (130)..(130): Xaa is R, K or is optionally absent
MXXXAXEAIEAALADFXKXXNXKDAAXVASKYMDXAXIFPLDMAXVDGRQXXQXLWQGLXDXGVSXXKXTXXXVQXSGDFAXESGXFSLKGPXXDSKLVXIAGXYVEVWRKGQXGGWKLYXTIANLXPAX

SEQ ID NO: 3 - protein, 130 aa, Artificial Sequence
  Synthetic
  MISC_FEATURE (1)..(1): is optionally absent
  MISC_FEATURE (2)..(2): Xaa is D, G, L or is optionally absent
  MISC_FEATURE (3)..(3): Xaa is Q or P
  MISC_FEATURE (4)..(4): Xaa is S or T
  MISC_FEATURE (6)..(6): Xaa is H or K
  MISC_FEATURE (19)..(19): Xaa is A or V
  MISC_FEATURE (37)..(37): Xaa is A or V
  MISC_FEATURE (45)..(45): Xaa is R or P
  MISC_FEATURE (76)..(76): Xaa is K or E
  MISC_FEATURE (100)..(100): Xaa is D or G
  MISC_FEATURE (127)..(127): Xaa is D or G
  MISC_FEATURE (129)..(129): is optionally absent
  MISC_FEATURE (130)..(130): Xaa is R, K or is optionally absent
MXXXAXEAIEAALADFVKXYNSKDAAGVASKYMDDAXIFPLDMAXVDGRQNIQKLWQGLMDMGVSEPKFTTLNVQXSGDFAFESGSFSLKGPGKDSKLVXIAGIYVEVWRKGQDGGWKLYRTIANLXPAX

SEQ ID NO: 4 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDVAGKYVVVWRKGQDGGWKLYRTISNLDPAK

SEQ ID NO: 5 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDVAGKYVEVWRKGQDGGWKLYRTISNLDPAK

SEQ ID NO: 6 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDIAFESGSFSLKGPGKDSKLVDVAGKYVEVWRKGQDGGWKLYRTISNLDPAK

SEQ ID NO: 7 - protein, 129 aa, Artificial Sequence
  Synthetic
GQIAKEAIEAALADFVKAYNSKDAAGVASKYMDDAAIFPLDMARVDGRQNIQKLWQGLMDMGMSELKLTTLDVQESGDFAFESGSFSLKGPGKDSKLVDVAGKYVEVWRKGQDGGWKLYRTISNPDPAK

SEQ ID NO: 8 - protein, 129 aa, Artificial Sequence
  Synthetic
GQSAKEAIEAVLADFVKAYNSKDAAGVVSKYMNDAAIFPLDMARVDGRQNIQKLWQGLMDMGVSELKLTTLDVQESGDFAFESGSFSLKGPGKDSKLVDVAGKYVEVWRKGQDGGWKLYCTISNLDPAK

SEQ ID NO: 9 - protein, 129 aa, Artificial Sequence; identical to SEQ ID NO: 4
  Synthetic

SEQ ID NO: 10 - protein, 129 aa, Artificial Sequence
  Synthetic
GQSAQEAIEAALADFVKAYNSKDAAGVASKYMDDAAIFPLDMARVDGRQNIQKLWQGLMDMGVSELKLTTLDVQESGDFAFESGSFSLKGPGKDSKLVDVAGKYVEVWRKGQDGGWKLYRTISNLDPAK

SEQ ID NO: 11 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDVAGKYVEVWRKGQDGDWKLYRTISNLDLAK

SEQ ID NO: 12 - protein, 129 aa, Artificial Sequence
  Synthetic
GQSAKEAIEAALADFVKAYNSKDAAGVASKYMDDAAIFPLDMARVDGRQDIQKLWQGLMDMGVSELKLTTLDVQESGDFAFESGSFSLKGPGKDSKLVDVAGKYVEVWRKGQDGGWKLYRTISNLDPAK

SEQ ID NO: 13 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDVAGKYVEVWRKGQDGGWKLYRTISNLNPAK

SEQ ID NO: 14 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDIAGKYVEVWRKGQDGGWKLYRTIANLDPAK

SEQ ID NO: 15 - protein, 129 aa, Artificial Sequence
  Synthetic
GQSAKEAIEAALADFVKAYNSKDAAGVASKYMDDAAIFPLDMARVDGRQNIQKLWQGLMDMGVSEVKLTTLDVQESGDFAFESGSFSAKGPGKDSKLVDMAGKYVEVWRKGQDGGWKLYRVIANLDPAK

SEQ ID NO: 16 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDIAGKYVEVWRKGQDGGWKLYRAIANLDPAK

SEQ ID NO: 17 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDIAGKYVEVWRKGQDGGWKLYRVIVNLDPAK

SEQ ID NO: 18 - protein, 129 aa, Artificial Sequence
  Synthetic
GQSAKEAIEAALADFVKAYNSKDAAGVASKYMDDAPIFPLDMARVDGRQNIQKLWQGLMDMGVSEPKLTTLDVQESGDFPFESGSFSLKGPGKDSKLVDIAGKYVEVWRKGQDGGWKLYRTIANLDPAK

SEQ ID NO: 19 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 20 - protein, 129 aa, Artificial Sequence
  Synthetic
GKSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 21 - protein, 129 aa, Artificial Sequence
  Synthetic
GQSAKEAIEAALADFVKAYNSKDAAGVASKYMDDAAIFPLDMARVDGRRNIQKLWQGLMDMGVSEPKLTTLDVQESGDFAFESGSFSLKGPGKDSKLVDIAGKYVEVWRKGQDGGWKLYRTIANLDPAK

SEQ ID NO: 22 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 23 - protein, 129 aa, Artificial Sequence
  Synthetic
GQSAKEAIEAALADFVKAYNSKDAAGVASKYMDDAAIFPLDMARVDGRQNILKLWQGLMDMGVCELKFTTLDVQESGDFAFESGSFSLKGPGKDSKLVDIAGKYVEVWRKGQDGGWKLYRTIANLDPAK

SEQ ID NO: 24 - protein, 129 aa, Artificial Sequence
  Synthetic
GQSAKEAIEAALADFVKVYNSKDAAGVASKYMDDAAIFPLDMAPVDGRQNIQKLWQGLMDMGVSGVKLTTLNVQESGDFAFESGSFSLKGPGKDSKLVDIAGIYVEVWRKGQDGGWKLYRTIANLDPAK

SEQ ID NO: 25 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDIKLVDIAGKYVEVWRKGQDGGWKLYRTIANLDPAK

SEQ ID NO: 26 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSISLKGPGKDSKLVDIAGKYVEVWRKGQDGGWKLYRTIANLDPAK

SEQ ID NO: 27 - protein, 129 aa, Artificial Sequence
  Synthetic
GQSAKEAIEAALADFVKAFNGKDAADVASKYMDDAAIFPLDMARVDGRQNIQKLWQGLMDTGVSEPKFTTLVVQESGDFAFESGSFSLKGPGPDSKLVDIAGKYVEVWRKGQDGGWKLYHTIANLDPAK

SEQ ID NO: 28 - protein, 129 aa, Artificial Sequence
  Synthetic
GQSAKEAIEAALADFVKAYNSKDAAGVASKYMDDAAIFPLDMAPVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 29 - protein, 129 aa, Artificial Sequence
  Synthetic
DQSAKEAIEAALADFVKVYNSKDAAGVASKYMDDAAIFPLDMAPVDGRQNIQKLWQGLMDMGVSEPKFTTLNVQKSGDFAFESGSFSLKGPGKDSKLVDIAGIYVEVWRKGQDGGWKLYRTIANLDPAK

SEQ ID NO: 30 - protein, 126 aa, Artificial Sequence
  Synthetic
QSAKEAIEAALADFVKVYNSKDAAGVASKYMDDAAIFPLDMAPVDGRQNIQKLWQGLMDMGVSEPKFTTLNVQKSGDFAFESGSFSLKGPGKDSKLVGIAGIYVEVWRKGQDGGWKLYRTIANLGP

SEQ ID NO: 31 - protein, 129 aa, Artificial Sequence
  Synthetic
DQSAKEAIEAALADFVKVYNSKDAAGVASKYMDDAVIFPLDMAPVDGRQNSQKLWQGLMDMGVSEPKFTTLNVQKSGDFAFESGSFSLKGPGKDSKLVDIAGIYVEVWRKGQDGGWKLYRTIANLDPAK

SEQ ID NO: 32 - protein, 129 aa, Artificial Sequence
  Synthetic
GQSAKEAIEAALADFVKAYNSKDAAGVASKYMDGAAIFPLDMAPVDGRQNIQKLWQGLIDMGVSGVKLTTLDVQESGDFAFESGSFSLKGPDKDSKLVDIAGKYVEVWRKGQDGGWKLYRTIANLDPAK

SEQ ID NO: 33 - protein, 129 aa, Artificial Sequence
  Synthetic
GQSAKEAIEAALADFLKAYNSKDAAGVASKYMDDAAIFPLDMARVDGRQYIQRLWQGLMDMGVSEPKFTTLNVQESGDFAFESGSFSLKGPGKDSKLVDIAGIYVEVWRKGQDGGWKLYRTIANLDPAK

SEQ ID NO: 34 - protein, 129 aa, Artificial Sequence
  Synthetic
LPTAHEAIEAALADFVKVYNSKDAAGVASKYMDDAVIFPLDMARVDGRQNIQKLWQGLMDMGVSEPKFTTLNVQESGDFAFESGSFSLKGPGKDSKLVDIAGIYVEVWRKGQDGGWKLYRTIANLDPAR

SEQ ID NO: 35 - protein, 129 aa, Artificial Sequence
  Synthetic
LPTAHEAIEAALADFVKVYNSKDAAGVASKYMDDAVIFPLDMARVDGRQNIQKLWQGLMDMGVSEPKFTILNVQESGDFAYESGSFSLKGPGKDSKLVDIAGIYVEVWRKGQDGGWKLYRTIANLDPAK

SEQ ID NO: 36 - protein, 129 aa, Artificial Sequence
  Synthetic
GQSAKEAIEAALADFVKAYNSKDAAGVASKYMDDAAIFPLDMAPVDGRQNIQKLWQGLMDMGVSEPKFTTMNVQESGDFAFESGRFSLKGPGKDSKLVDIAGKYVEVWRKGQGGGWKLYRTIANLDPAK

SEQ ID NO: 37 - protein, 129 aa, Artificial Sequence; identical to SEQ ID NO: 28
  Synthetic

SEQ ID NO: 38 - protein, 129 aa, Artificial Sequence
  Synthetic
GQSAKEAIEAALADFVKAYNSKDAAGVASKYMDDAAIFPLDMAPVDGRQNIQKLWQGLIDMGVSGVKLTTLDVQESGDFAFESGSFSLKGPGKDSKLVDNAGKYVEVWRKGQDGGWKLYRTIANLDPAK

SEQ ID NO: 39 - protein, 129 aa, Artificial Sequence
  Synthetic
DQSAKEAIEAALADFVKVYNSKDAAGVASKYMDDAVIFPLDMARVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 40 - protein, 129 aa, Artificial Sequence
  Synthetic
GQSAKEAIEAALADFVKAYNSKDAAGVASKYMDDAAIFPLDMAPVDGRQNIQKLWQGLMDMGVSEPKFTTLNVQESGDFAFESGSFRLKGPGKDSKLVDIAGIYVEVWRKGQDGGWKLYRTIANLDPAK

SEQ ID NO: 41 - protein, 129 aa, Artificial Sequence
  Synthetic
GQSAKEAIEAALADFVKVYNSKDAAGVASKYMDDAAIFPLDMAPVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 42 - protein, 129 aa, Artificial Sequence
  Synthetic
DQSAKEAIEAALADFVKVYNSKDAAGVASKYMDDAVIFPLDMARVDGRQNIQKLWQGLMDMGVSEPKFTTLNVQESGDFAFESGSFSLKGPGKDSKLVDIAGIYVEVWRKGQDGGWKLYRTTANLDPAK

SEQ ID NO: 43 - protein, 129 aa, Artificial Sequence
  Synthetic
LPTAHEAIEAALADFVKVYNSKDAAGVASKYMDDAVIFPLDMARVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 44 - protein, 129 aa, Artificial Sequence
  Synthetic
GQSAKEAIEATLADFVKAYNSKDAAGVASKYMDDAAIFPLDMAPVGGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 45 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQENGDFAFESGSFSLKGPGKDSKLVDIAGKYVEVWRKGQGGGWKLYRTIANLDPVK

SEQ ID NO: 46 - protein, 129 aa, Artificial Sequence
  Synthetic
GQSAKEAIEAALADFVKAYNSKDAAGVASKYMDDAAIFPLDMAPVDGRQNIQKLWQGLMDMGVSEPKLTTLNVQESGDFAFESGSFSLKGPGKDSKLVDIAGIYVEVWRKGQDGGWKLYRTIANLDPAK

SEQ ID NO: 47 - protein, 129 aa, Artificial Sequence
  Synthetic
GQSAKEAIEAALADFVKAYNSKDAAGVASKYMDDAAIFPLDMAPVDGRQNIQKLWQGLMDMGVSEPKFTTLNVQESGDFAFESGSFSLKGPGKDSKLVDIAGIYEEVWRKGQDGGWKLYRTIANLDPAK

SEQ ID NO: 48 - protein, 129 aa, Artificial Sequence
  Synthetic
GQSAKVAIEAALADFVKVYKSKDVAGVASKYMDDAVIFPLDMAPVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 49 - protein, 129 aa, Artificial Sequence
  Synthetic
GQSAKEVIEAALADFVKAYNSKDAAGVASKYMDDAAIFPLDMAPVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 50 - protein, 129 aa, Artificial Sequence
  Synthetic
DQSAKEPIEAALADFVKGYNSKDAAGVASKYMDDAVIFPLDMARVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 51 - protein, 129 aa, Artificial Sequence; identical to SEQ ID NO: 14
  Synthetic

SEQ ID NO: 52 - protein, 129 aa, Artificial Sequence
  Synthetic
GQSAKEAIEAALADFVKAYNSKDAAGVASKYMDDAAIFPLDMARVDGRQNIQKLWQGLMDMGVSELKSTTLDVQESGDFAFESGSFSLKGPGKDSKLVDVAGKYVVVWRKGQDGGWKLYRTISNLDPAK

SEQ ID NO: 53 - protein, 129 aa, Artificial Sequence
  Synthetic
GQSAKEAIEAALADFVKAYNSKDAAGVASKYVDDAAIFPLDMARVDGRQNIQKLWQGLMDMGVSELKSTTLDVQESGDFAFESGSFSLKGPGKDSKLVDVAGKYVVVWRKGQDGGWKLYRTISNLDPAK

SEQ ID NO: 54 - protein, 129 aa, Artificial Sequence
  Synthetic
GQSAKEAIEAALADFVKAYNSKDAAGVASKYMDDAAIYPLDMARVDGRQNIQKLWQGLMDMGVSELKSTILDVQESGDFAFESGSFSLKGPGKDSKLVDVAGKYVVVWRKGQDGGWKLYRTISNLDPAK

SEQ ID NO: 55 - protein, 129 aa, Artificial Sequence
  Synthetic
GQSAKEAIEAALADFVKAYNSKDAAGVASKYMDDAAIFPLDMARVDGRQNIQRLWQGLMDMGVSELKSTTLDVQESGDFAYESGSFSLKGPGKDSKLVDVAGKYVVVWRKGQDGGWKLYRTISNLDPAK

SEQ ID NO: 56 - protein, 129 aa, Artificial Sequence
  Synthetic
GQSAKEAIEAALADFVKAYNSKDAAGVASKYMDDAAIFPLDMARVDGRQNIQKLWQGLMDMGVSELKSTTLDVQESGDFAFESGSFSLKGPGKDSKLVDVAGKYVEVWRKGQDGGWKLYRTISNLDPAK

SEQ ID NO: 57 - protein, 129 aa, Artificial Sequence; identical to SEQ ID NO: 26
  Synthetic

SEQ ID NO: 58 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDNKLVDIAGKYVEVWRKGQDGGWKLYRTIANLDPAK

SEQ ID NO: 59 - protein, 129 aa, Artificial Sequence; identical to SEQ ID NO: 22
  Synthetic

SEQ ID NO: 60 - protein, 129 aa, Artificial Sequence
  Synthetic
GQSAKEAIEAALADFVKAYNSKDAAGLASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 61 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTALDVQESGDFAFESGSFSLKGPGKDSKLVDIAGKYVEVWRKGQDGGWKLYRTIANLDPAK

SEQ ID NO: 62 - protein, 129 aa, Artificial Sequence
  Synthetic
GQSAKEAIEAALADFVKSYNSKDAAGVASKYMDDAAIFPLDMAPVDGRQNIQKLWQGLMDMGVSGLKLTTLDVQESGDFAFESGSFSLKGPGRDSKLVDITGKYVEVWRKGQDGGWKLYRTIANLDPAK

SEQ ID NO: 63 - protein, 129 aa, Artificial Sequence; identical to SEQ ID NO: 19
  Synthetic

SEQ ID NO: 64 - protein, 129 aa, Artificial Sequence
  Synthetic
GQIAKEAIEAALADFVKAYNSKDAAGVVSKYMDDAAIFPLDMAPVDGRQNIQKLWQGLMDMGVSGVKLTTLDVQESGDFAFESGSFSLKGPGKDSKLVDIAGKYVEVWRKGQDGDWKLYRTIANLDPAK

SEQ ID NO: 65 - protein, 129 aa, Artificial Sequence
  Synthetic
GQSAKEAIEAALADFVKAYNSKDAAGVASKYTDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 66 - protein, 129 aa, Artificial Sequence
  Synthetic
GQSAKEAIEAALADFVKVYNSKDAAGVAGKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 67 - protein, 129 aa, Artificial Sequence
  Synthetic
GQSAKEAIEAALADFVKAYNSKDAAGVASKYMDDAAIFPLDMARVDGRQDIQKLWQGLMDMGVSEPKLTTLDVQESGDFAFESGSFSLKGPGKDSKLVDIAGKYVEVWRKGQDGGWKLYRTIANLDPAK

SEQ ID NO: 68 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGKDGGWKLYRTIANLDPAK

SEQ ID NO: 69 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQNGGWKLYRTIANLDPAK

SEQ ID NO: 70 - protein, 129 aa, Artificial Sequence
  Synthetic
GQNAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 71 - protein, 129 aa, Artificial Sequence
  Synthetic
AQSAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGRKLYRTIANLDPAK

SEQ ID NO: 72 - protein, 128 aa, Artificial Sequence
  Synthetic
GQSAKEAIEAALADFVKAYNSKDAAGVASKYMDDAAIFPLDMAPVDGHQNIQKLWQGLMDMGVSGVKLTTLDVQESGDFAFESGSFSLKGPGKDSKLVDIAGKYVEVWRKGQDGDWKLYRTIANLARR

SEQ ID NO: 73 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDNAGKYVEVWRKGQDGGWKLYRTIANLDPAK

SEQ ID NO: 74 - protein, 129 aa, Artificial Sequence
  Synthetic
GQSAKEAIEAALADFVKAYNSKDAAGVARKYMDDAAIFPLDMARVDGRQNIQKLWQGLMDMGVSEPKLTTLDVQESGDFAFESGSFSLKGPGKDSKLVDNAGKYVEVWRKGQDGGWKLYRTIANLDPAK

SEQ ID NO: 75 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGNFSLKGPGKDSKLVDIAGKYVEVWRKGQDGGWKLYRTIANLDPAK

SEQ ID NO: 76 - protein, 129 aa, Artificial Sequence
  Synthetic
GQSAKEAIEAALADFVKAYNSKDAAGVASKYMDDAAIFPLDMARVDGRQNIQKLWQGLLDMGVSEPKLTTLDVQESGDFAFESGSFSLKGPGKDSKLVDIAGKYVEVWRKGQDGGWKLYRTIANLDPAK

SEQ ID NO: 77 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFVFESGSFSLKGPGKDSKLVDIAGKYVEVWRKGQDGGWKLYRTIANLDPAK

SEQ ID NO: 78 - protein, 129 aa, Artificial Sequence
  Synthetic
GQIAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 79 - protein, 129 aa, Artificial Sequence
  Synthetic
GQSAKEAIEAALADFVKAYNSKDAAGVASKYMDDAAIFPLDMARVDGRQNIQKLWQGLMDTGVSEPKLTTLDVQESGDFAFESGSFSLKGPGKDSKLVDIAGKYVEVWRKGQDGGWKLYRTIANLDPAK

SEQ ID NO: 80 - protein, 129 aa, Artificial Sequence
  Synthetic
GQSAKEAIEAALADFVKAYNSKDAAGVASKYMDDAAIFPLDMARVDGRQNIQKLWQGLMDMCVSEPKLTTLDVQESGVFAFESGSFSLKGPGKDSKLVDIAGKYVEVWRKGQDGGWKLYRTIANLDPAK

SEQ ID NO: 81 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVGVWRKGQDGGWKLYRTIANLDPAK

SEQ ID NO: 82 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFTFESGSFSLKGPGKDSKLVDIAGKYVEVWRKGQDGGWKLYRTIANLDPAK

SEQ ID NO: 83 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFRLKGPGKDSKLVDIAGKYVEVWRKGQDGGWKLYRTIANLDPAK

SEQ ID NO: 84 - protein, 129 aa, Artificial Sequence
  Synthetic
GQSAKEAIESALADFVKVYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 85 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLADIAGKYVEVWRKGQDGGWKLYRTIANLDPAK

SEQ ID NO: 86 - protein, 129 aa, Artificial Sequence
  Synthetic
GQSAKEAIEAALADFVKAYNSKDAAGVASKYMDDAAIFPLDMAPVDGRQNIQKLWQGLMDLGVSEPKFTTLDVQESGDFAFESGSFSLKGPGQDSKLVDIAGKFVEVWRKGQDGGWKLYRTIANLDPAK

SEQ ID NO: 87 - protein, 129 aa, Artificial Sequence
  Synthetic
GQSAKETIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 88 - protein, 129 aa, Artificial Sequence
  Synthetic
GQSAKEAIEAALADFVKAYNSKDAAGVASKYMDDAAIFPLGMARVDGRQNIQKLWQGLMDMGVSELKSTTLDVQESGDFAFESGSFSLKGPGKDSKLVDVAGKYVVVWRKGQDGGWKLYRTISNLDPAK

SEQ ID NO: 89 - protein, 129 aa, Artificial Sequence
  Synthetic
GQSAKEAIEAALADLVKAYNSKDAAGVASKYMDDAAIFPLDMARVDGRQNIQKLWQGLMDMGVSELKSTTLDVQESGDFAFESGSFSLKGPGKDSKLVDVAGKYVVVWRKGQDGGWKLYRTISNLDPAK

SEQ ID NO: 90 - protein, 128 aa, Artificial Sequence
  Synthetic
GQSAKEAIEAALADFVKAYNSKDAAGVASKYMDDAAIFPLDMARVDGRQNIQKLWQGLMDMGVSELKSTTLDVQESGDFAFESGSFSLKGPGKDSKLVDVAGKYVEVWRKGQDGGWKLYRTINLDPAK

SEQ ID NO: 91 - protein, 129 aa, Artificial Sequence
  Synthetic
GQSAKEAIEAALADFVKAYNSKDAAGVASKYMDDAAIFPLDMVRVDGRQNIQKLWQGLMDMGVSELKSTTLDVQESGDFAFESGSFSLKGPGKDSKLVDVAGKYVEVWRKGQDGGWKLYRTISNLDPAK

SEQ ID NO: 92 - protein, 129 aa, Artificial Sequence
  Synthetic
GQSAKEAIEAALADFVKAYNSKDAAGVASKYLDDAAIFPLDMARVDGRQNIQKLWQGLMDMGVSGPKFTTLDVQESGDFAFESGSFSLKGPGKDSKLVDVAGKYVVVWRKGQDGGWKLYRTISNLDPAK

SEQ ID NO: 93 - protein, 129 aa, Artificial Sequence
  Synthetic
GQSAKEAIEAALADFVKAYNSKDAAGVASKYMDDAAIFPLDMARVDGRQDIQKLWQGLMDMGVSELKSTTLDVQESGDFAFESGSFSLKGPGKDSKLVDVAGKYVVVWRKGQDGGWKLYRTISNLDPAK

SEQ ID NO: 94 - protein, 129 aa, Artificial Sequence; identical to SEQ ID NO: 4
  Synthetic

SEQ ID NO: 95 - protein, 129 aa, Artificial Sequence
  Synthetic
GQSAKEAIEAALADFVKAYNSKDAAGVASKYMDDAAIFPLDMARVDGRQNIQKLWQGLMDMGVSELKSTTLDVQESGDFAFESGSFSLKGPGKDSKMVDVAGKYVVVWRKGQDGGWKLYRTISNLDPAK

SEQ ID NO: 96 - protein, 129 aa, Artificial Sequence
  Synthetic
GQSAKEAIEAALADFVKAYNSKDAAGVASKYMDDAAIFPLDMARVDGRQNIQKLWQGLMDMGVSELKSTTLDVQESGDFAFESGSFSLKGPGKDSKLVDVVGKYVVVWRKGQDGGWKLYRTISNLDPAK

SEQ ID NO: 97 - protein, 129 aa, Artificial Sequence
  Synthetic
GQSAKEAIEAALADFVKAYNSKDAAGVASKYMDDAAIFPLDMARVDGRQNIQKLWQGLMDMGVSELKSTTLDVQESGDFAFESGSFSLKGPGKDSKLADVAGKYVVVWRKGQDGGWKLYRTISNLDPAK

SEQ ID NO: 98 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQDGGWKLYRTIANLGPAK

SEQ ID NO: 99 - protein, 129 aa, Artificial Sequence
  Synthetic
GQSAKEAIEAALADFVKAYNSKDAAGVASKYMDDAAIFPLDMARVDGRQNIQKLWQGLMDMGVSELKSTTLDVQESGDFAFESGSFSLKGPGKDGELVDVAGKYVVVWRKGQDGGWKLYRTISNLDPAK

SEQ ID NO: 100 - protein, 129 aa, Artificial Sequence
  Synthetic
GQSAKEAIEAALADFVKAYNSKDAAGVASKYMDDAAIFPLDMARVDGRQNIQKLWQGLMDMGMSELKSTTLDVQESGDFAFESGSFSLKGPGKDSKLVDVAGKYVVVWRKGQDGGWKLYRTISNLDPAK

SEQ ID NO: 101 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKSTTLDVQESGDFAFESGSFSLKGPGKDSKLVDIAGKYVEVWRKGQDGGWKLYRTIANLDPAK

SEQ ID NO: 102 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKFTTLVVQESGDFAFESGSFSLKGPGKDSKLVDIAGKYVVVWRKGQDGGWKLYRTISNLDPAK

SEQ ID NO: 103 - protein, 129 aa, Artificial Sequence
  Synthetic
GQSAKEAIEAALADFVKAYNSKDAAGVASKYMDDAAIFPLDMARVDGRQNILKLWQGLMDMGVSELKSTTLDVQESGDFAFESGSFSLKGPGKDSKLVDVAGKYVVVWRKGQDGGWKLYRTISNLDPAK

SEQ ID NO: 104 - protein, 129 aa, Artificial Sequence
  Synthetic
GQSAKEAIEAALADFVKAYNGKDAAGVASKYMDDAAIFPLDMARVDGRQNIQKLWQGLMDMGVSELKSTTLDVQESGDFAFESGSFSLKGPGKDNKLVDVAGKYVEVWRKGQDGGWKLYRTISNLDPAK

SEQ ID NO: 105 - protein, 129 aa, Artificial Sequence
  Synthetic
GQSAKEAIEAALADFVKAYNSKDAAGVASKYIDDAAIFPLDMARVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 106 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQDGGWKLYRTIANLDPSK

SEQ ID NO: 107 - protein, 129 aa, Artificial Sequence
  Synthetic
GQSAKEAIEAALADFVKAYNSKDAADVASKYMDDAAIFPLDMAPVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 108 - protein, 129 aa, Artificial Sequence
  Synthetic
GQRAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 109 - protein, 129 aa, Artificial Sequence
  Synthetic
GQSAKEAIEAALADFVKAYNSKDAAGVASKYMDDAAIFPLDMASVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 110 - protein, 129 aa, Artificial Sequence
  Synthetic
GQSSKEAIEAALADFVKAYNSKDAAGVANKYMDDAAIFPLDMARVDGRQNIQKLWQGLMDMGVSEPKLTTLDVQESGDFAFESGSFSLKGPGKDSKLVDIAGKYVEVWRKGQDGGWKLYRTIANLDPTK

SEQ ID NO: 111 - protein, 127 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLPG

SEQ ID NO: 112 - protein, 128 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGDSKLVDIAGKYVEVWRKGQDGGWKLYRTIANLDPAK

SEQ ID NO: 113 - protein, 129 aa, Artificial Sequence
  Synthetic
GQSAKEAIEAALAEFVKAYNCKDAAGVASKYMDDAAIFPLDMARVDGRQNIQKLWQGLMDMGVSEPELTTLDVQESGDFAFESGSFSLKGPGKDSKLVDIAGKYVEVWRKGQDGGWKLYRTIANLDPAK

SEQ ID NO: 114 - protein, 129 aa, Artificial Sequence
  Synthetic
SQSAKETIEAALADFVKAYNSKDAAGVASKYMDDAEIFPLDMARVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 115 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDVAGKYVMVWRKGQDGGWKLYRTISNLDPAK

SEQ ID NO: 116 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLGVQESGDFAFESGSFSLKGPGKDSKLVDIAGKYVEVWRKGQDGGWKLYRTIANLDPAK

SEQ ID NO: 117 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQDGGWKLYRTISNLDPAK

SEQ ID NO: 118 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGNFSLKGPGKDSKLVDIAGKYVEVWRKGQDGGWKLYRTIANLDPAK

SEQ ID NO: 119 - protein, 129 aa, Artificial Sequence
  Synthetic
GQSAKEAIEAALADFVKAYNSKDAAGVASKYMDDAAIFPLDMARVDGRQNIQKLWQGLTDMGVSELKLTTLDVQESGDFAFESGSFSLKGPGKDSKLVDVAGKYVEVWRKGQDGGWKLYRTISNLDPAK

SEQ ID NO: 120 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGYFAFESGSFSLKGPGKDSKLVDIAGKYVEVWRKGQDGGWKLYRTIANLDPAK

SEQ ID NO: 121 - protein, 129 aa, Artificial Sequence
  Synthetic
GQSAEEAIEAALAEFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 122 - protein, 129 aa, Artificial Sequence
  Synthetic
GQSAKEAIEAALADFVKAYNSKDAAGVVSKYMDDAAIFPLDMARVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 123 - protein, 129 aa, Artificial Sequence
  Synthetic
GQSAKEAIKAALADFVKAYNSKDAAGVASKYMDDAAIFPLDMARVDGRQNIQKLWQGLMDMGVSELKLTTLDVQESGDFAFESGSFSLKGPGKDSKLVDVAGKYVEVWRKGQDGGWKLYRTISNLDPAK

SEQ ID NO: 124 - protein, 129 aa, Artificial Sequence
  Synthetic
GQSAKEAIEAALADFVKAYNSKDAAGVASKYMDDAAIFPLDMARVVGRQNIQKLWQGLMDMGVSEPKFTTLDVQESGDFAFESGSFSLKGPGQDSKLVDIAGKYVEVWRKGQDGGWKLYRTIANLDPAK

SEQ ID NO: 125 - protein, 129 aa, Artificial Sequence
  Synthetic
GQSAKEAIEAALADFVKGYNPKDGAGVASKSMDDAPIFPPDMARVDGRQNIQKLWQGLMDTGVSEPKFTTLDVQESGDFAFESGSFSLKGPGPDSKLVDIAGKYVVVWRKGQDGGWKLYRTISNLDPAK

SEQ ID NO: 126 - protein, 129 aa, Artificial Sequence
  Synthetic
GQSAKEAIEAALADFVKAYNSKDAAGVASKYMDGAAIFPLDMARVDGRQNIQKLWQGLMDMGVSELKLTTLDVQESGDFAFESGSFSLKGPGKDSKLVDVAGKYVVVWRKGQDGGWKLYRTISNLDPAK

SEQ ID NO: 127 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVGVAGKYVEVWRKGQDGGWKLYRTISNLDPAK

SEQ ID NO: 128 - protein, 129 aa, Artificial Sequence
  Synthetic
GQSAKEAIEAALADFLKGYNPKDGAGVASKYMDDAPIFPPDMAPVDGPQNILKLWQGLMDMGVSGPKFTTLVVQESGDFAFESGSFSPKGPGKDSKLVDIAGKYVVVWRKGQDGGWKLYRTISNLDPAK

SEQ ID NO: 129 - protein, 129 aa, Artificial Sequence
  Synthetic
GQSAKEAIEAALADFAKVYNGKDGAGVASKSMDDAPIFPPDMAPVDGPQNILKLWQGLMDMGVSEPKFTTLVVQESGDFAFESGSFSVKGPGTDSKLVDIAGKYVVVWRKGQDGGWKLYRTISNLDPAK

SEQ ID NO: 130 - protein, 129 aa, Artificial Sequence
  Synthetic
GQSAKEAIEAALADFVKGYNRKDGAGVASKSMDDAPIFPLDMAPVDGPQNILKLWQGLMDTGVSEPKFTTLVVQESGDFAFESGSFSVKGPGPDSKLVDIAGKYVVVWRKGQDGGWKLYRTISNLDPAK

SEQ ID NO: 131 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTMDVQESGDFAFESGSFSLKGPGKDSKLVDVAGKYVVVWRKGQDGGWKLYRTISNLDPAK

SEQ ID NO: 132 - protein, 129 aa, Artificial Sequence
  Synthetic
GQSAKEAIEAALADFVKAYNSKDAAGVASKYMDDAAIFPLDMALVDGRQNIQKLWQGLMDMGVSGVKLTTLDVQESGDFAFEGGSFSLKGPGKDSKLVDIAGKYVEVWRKGQDGGWKLYRTIANLDPAK

SEQ ID NO: 133 - protein, 129 aa, Artificial Sequence
  Synthetic
GQSAKEAIEAALADFVKAYNSNDATGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 134 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGNDSKLVDIAGKFVEVWRKGQDGGWKLYRTIANLDPAK

SEQ ID NO: 135 - protein, 129 aa, Artificial Sequence
  Synthetic
GQSAKEAIEAALADFVKAYNSKDAAGVASKYMDDAAIFPLDMAPVDGRQNIQKLWQGLKDMGVSGVKLTTLDVQESGDFAFESGSFSLKGPGKDSKLVDIAGKYVEVWRKGQDGGWKLYRTIANLDPAK

SEQ ID NO: 136 - protein, 129 aa, Artificial Sequence
  Synthetic
GQSAKEAIEAALADFVKAYNSKDAAGVASKYMDDAAIFPLDMARVDGRQNIQKLWQDLMDMGVSEPKLTTLDVQESGDFAFESGSFSLKGPGKDSKLVDIAGKYVEVWRKGQDGGWKLYRTIANLDPAK

SEQ ID NO: 137 - protein, 129 aa, Artificial Sequence
  Synthetic
GQSAKEAIEAALADFVKAYNSKDAAGVASKYMDDAAIFPLDMAPVDGCQNIQKLWQGLMDMGVSGVKLTTLDVQESGDFAFESGSFSLKGPGKDSKLVDIAGKYVEVWRKGQDGGWKLYRTIANLDPAK

SEQ ID NO: 138 - protein, 129 aa, Artificial Sequence
  Synthetic
GQSAKEAIEAALADFVKAYNSKDAAGVASKYTDDAAIFPLDMARVDGRQNIQKLWQGLMDMGVSEPKLTPLDVQESGDFAFESGSFSLKGPGKDSKLVDIAGKYVEVWRKGQDGGWKLYRTIANLDPAK

SEQ ID NO: 139 - protein, 129 aa, Artificial Sequence
  Synthetic
GQSAKEAIEAALADFVKACNSKDAAGVASKYMDDAAIFPLDMARVDGRQNIEKLWQGLMDMGVSEPKLTTLDVQESGDFAFESGSFSLKGPGKDSKLVDIAGKYVEVWRKGQDGGWKLYRTIANLDPAK

SEQ ID NO: 140 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKSPGKDSKLVDIAGKYVEVWRKGQDGGWKLYRTIANLDPAK

SEQ ID NO: 141 - protein, 129 aa, Artificial Sequence
  Synthetic
GQSVKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 142 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDVAFESGSFSLKGPGKDSKLVDIAGKYVEVWRKGQDGGWKLYRTIANLDPAK

SEQ ID NO: 143 - protein, 129 aa, Artificial Sequence
  Synthetic
GQSAKEAIEAALADFVKAYNSKDAAGVASKYKDDAAIFPLDMARVDGRQNIQKLWQGLMDMGVSEPKLTTQDVQESGDFAFESGSFSLKGPGKDSKLVDIAGKYVEVWRNGQDGGWKLYRTIANLDPAK

SEQ ID NO: 144 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGRDGGWKLYRTIANLDPAK

SEQ ID NO: 145 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGSGKDSKLVDIAGKYVEVWRKGQDGDWKLYRTIANLDPAK

SEQ ID NO: 146 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGPDSKLVDIAGKYVEVWRKGPDGGWKLYRTIANLDPAK

SEQ ID NO: 147 - protein, 129 aa, Artificial Sequence
  Synthetic
GQSAKEAIEAALADFVKAYNSKDAAGVASKYMDDAAIFPLDMTPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 148 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANPDPAK

SEQ ID NO: 149 - protein, 129 aa, Artificial Sequence
  Synthetic
GQSAKEAIEAALADFVKAYNSKDAAGVASKYKDDAAIFPLDMAPVDGRQNIQKLWQGLMDMGVSGVKSTTLDVQESGDFAFESGSFSLKGPGKDSKLVDIAGKYVEVWRKGQDGGWKLYRTIANLDPAK

SEQ ID NO: 150 - protein, 129 aa, Artificial Sequence
  Synthetic
GQSAKEAIEAALADFVKAYNSKDAAGVASKYMDDAAIFPLGMARVDGRQNIQKLWQGLMDMGVSEPKLTTLDVQESGDFAFESGSFSLKGPGKDSKLVDIAGKYVEVWRKGQDGGWKLYRSIANLDPAK

SEQ ID NO: 151 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANPDPAE

SEQ ID NO: 152 - protein, 129 aa, Artificial Sequence
  Synthetic
GQSAKEAIEAALADFVKAYNSKDAAGVASKYMDDAAIFPLDMAPVDGRQNIQKLWQGLMDMGMSGVKLTTLDVQESGDFAFESGSFSLKGPGKDSKLVDIACKYVEVWRKGQDGGWKLYRTIANLDPAK

SEQ ID NO: 153 - protein, 129 aa, Artificial Sequence
  Synthetic
GQSAKEAIEAVLADFVKAYNSMDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTITNLDPAK

SEQ ID NO: 154 - protein, 129 aa, Artificial Sequence
  Synthetic
GQSAKEAIEAALADFVKAYNSKDAAGVASKYMDDAAIFLLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 155 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPVKDSKLVDIAGKYVEVWGKGQDGGWKLYRTIANQDPAK

SEQ ID NO: 156 - protein, 129 aa, Artificial Sequence
  Synthetic
GQSAKEAIEAALADFVKAYNSNDAAGVASKYMDDPAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 157 - protein, 129 aa, Artificial Sequence
  Synthetic
GQSAKEAVEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 158 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTSLDVQESGDFAFESGSFSLKGPGKDSKLVDIAGKYVEVWRKGQDGGWKLYRIIANLDPAK

SEQ ID NO: 159 - protein, 129 aa, Artificial Sequence
  Synthetic
GQSAKEAIEAALADFVKAYNSKDTTGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 160 - protein, 129 aa, Artificial Sequence
  Synthetic
GQSAKEAIEAALADFVKAYNSKDAAGVASKYMDDASIFPLDMARVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 161 - protein, 129 aa, Artificial Sequence
  Synthetic
GQSAKEAIEAALADFVKAYNSNDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 162 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPDKDSKLVDIAGKYVEVWRKGQDGGWKLYRTIANLDPAK

SEQ ID NO: 163 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDVAFESGSFSLKGPGKDSKLVDIAGKYVEVWRKGQDGGWKLYRTIANLDPAK

SEQ ID NO: 164 - protein, 129 aa, Artificial Sequence
  Synthetic
GQSAKEAIEAALADFVKAYNSKDAAGLASKYMDDAAIFPLDMARVDGRQNIQKLWQGLMDMGVSEPKLTTLDVQESGDFAFESGSFSLKGPGKDSKLVDIAGIYVEVWRKGQDGGWKLYRTIANLDPAK

SEQ ID NO: 165 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDIAGKYVEVWRKGQDGGWTLYRIIANLDPAK

SEQ ID NO: 166 - protein, 129 aa, Artificial Sequence
  Synthetic
GQSAKEAIEAALADFVKAYNSKDAAGVASKYMDDAAIFPLDMAPVDGRQNIPKMWQGLMDMGVSGVKLTTLDVQESGDFAFESGSFSLKGPGKDSKLVDIAGKYVEVWRKGQDGGWKLYRTIANLDPAK

SEQ ID NO: 167 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGKDCKLVDIAGKYVKVWRKGQDGGWKLYRTIANLDPAK

SEQ ID NO: 168 - protein, 129 aa, Artificial Sequence
  Synthetic
GQSAKEAIEAALADSVKAYNSKDAAGVASKYMDDAAIFPLDMARVDGRQNIQKLWQGLMDMGVSEPKLTTLDVQECGDFAFESGSFSLKGPGKDSKLVDIAGKYVEVWRKGQDGGWKLYRTIANLDPAK

SEQ ID NO: 169 - protein, 129 aa, Artificial Sequence
  Synthetic
GQSAKEAIEAALADFVKAYNSKDAAGVASKYMDDAAIFPLDMAPVDGRQNIQKLLQGLMDMGVSGVKLTTLDVQESGDFAFESGSFSLKGPGKDSKLVDIAGKYVEVWRKGQYGGWKLYRTIANLDPAK

SEQ ID NO: 170 - protein, 129 aa, Artificial Sequence
  Synthetic
GQSAKEAIEAALADFVKAYNSKDAAGVASNYMDDAAIFPLDMAPVDGRQNIQKLWQGLMDMGVSGVKLTTLDVQESGDFAFESGSLSLKGPGKDSKLVDIAGKYVEVWRKGQDGGWKLYRTTANLDPAK

SEQ ID NO: 171 - protein, 129 aa, Artificial Sequence
  Synthetic
GQSAKEAIEAALADYVKAYNNKDAAGVASKYMDDAAIFPQDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 172 - protein, 129 aa, Artificial Sequence
  Synthetic
GQSAKEAIEAALADFVKAYNSKDAAGVASKYMDDAAIFPLDMAPVDGRQNIQKLWQGLMDMGVNGVKLTTLDVQESGDFTFVSGSFSLKGPGKDSKLVDIAGKYVEVWRKGQDGGWKLYRTIANLDPAK

SEQ ID NO: 173 - protein, 129 aa, Artificial Sequence
  Synthetic
GQSAKEAIEAALADFVKAYNSKDGAGVASKYMDDAPIFPLDMARVDGRQNIQKLWQGLMDTGVSEPKFTTLVVQESGDFAFESGSFSPKGPGTDSKLVDIAGKYVEVWRKGQDGGWKLYRTIANLEPAK

SEQ ID NO: 174 - protein, 129 aa, Artificial Sequence
  Synthetic
GQSAKEAIEAALADSVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 175 - protein, 129 aa, Artificial Sequence
  Synthetic
GQSAKEAIEAALADFVKAYNSKDAAGVASKYMDDAAIFPPDMAPVDGRQNIQKLWQGLMDMGVSGPKLTTLVVQESGDFAFESGSFSLKGPGTDSKLVDIAGKYVEVWRKGPDGGWKLYRTIANLDPAK

SEQ ID NO: 176 - protein, 129 aa, Artificial Sequence
  Synthetic
GQTAKEAIEAALADFVKAYNSKDAAGVASKYMDDAAIFPLDMAPADGRQNIQKLWQGLMDMGVSGVKLTTLDVQESGDFAFESGSFSLKGPGKDSKLVDIAGEYVEVWRKGQDGGWKLYRTIANLDPAK

SEQ ID NO: 177 - protein, 129 aa, Artificial Sequence
  Synthetic
GQSAKEAIEAALADFVKAYNSKDAAGVASKYMDDVAIFPLDMARVDGRQNIQKLWQGLMDMGVSEPKLTTLDVQESGDFAFESGSFSLKGPGKDSKLVDIAGEYVEVWRKGQDGGWKLYRTIANLDPAK

SEQ ID NO: 178 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFSLKGPGKDSKLVDVAGKYVEVWRKGQDGGWKLYRTIANLDPAK

SEQ ID NO: 179 - protein, 129 aa, Artificial Sequence
  Synthetic
GQSAKEAIEAALADFVKAYNSKDTAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 180 - protein, 129 aa, Artificial Sequence
  Synthetic
GQSAKEAIEAALADFVKVYNSKDAAGVASKYMDDAAIFPLDMAHVDGRQNIQKLWQGQMDMGVSGVKLTTLDVQESGDFAFESGSFSLKGPGKDSKLVDIAGKYVEVWRKGQDGGWKLYRTIANLDPAK

SEQ ID NO: 181 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFASESGSFSLKGPGKDSKLVDIAGKYVEVWRKGQDGGWKLYRTIANLDPAK

SEQ ID NO: 182 - protein, 129 aa, Artificial Sequence
  Synthetic
GQSAKEAIEAALADFVKAYNSKDAAGVASKYMDDAAIFPPDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 183 - protein, 129 aa, Artificial Sequence
  Synthetic
DQSAKEAIEAALADFVKVYNSKDAAGVASKYMDDAVIFPLDMARVDGRQNIQKLWQGLMDMGVSDPKFTTLNVQESGDFAFESGSFSLKGPGKDSKLVDIAGIYVEVWRKGQDGGLKLYRTIANLDPAK

SEQ ID NO: 184 - protein, 129 aa, Artificial Sequence; identical to SEQ ID NO: 36
  Synthetic

SEQ ID NO: 185 - protein, 129 aa, Artificial Sequence; identical to SEQ ID NO: 24
  Synthetic

SEQ ID NO: 186 - protein, 129 aa, Artificial Sequence
  Synthetic
GQSAKEAIEAALADFVKAYNSKDAAGVASKYMDDAAIFPLDMAPVDGRQNIQKLWQGLMDMGVSEPKFTTLNVQESGDFAFESGSFSLKGPGKDSKLVDIAGIYVEVWRKGQDGSWKLYRTIANLDPAN

SEQ ID NO: 187 - protein, 129 aa, Artificial Sequence
  Synthetic
GQSAKEAIEAALADFVKVYNSKDAAGVASKYMDDAVIFPMDMARVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 188 - protein, 129 aa, Artificial Sequence
  Synthetic
GQSAKVAIEAALADFVKVYNSKDVAGVASKYMDDAVIFPLDMARVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 189 - protein, 129 aa, Artificial Sequence
  Synthetic
GQSAKVAIEAALADFVKVYNSKDVAGVASKYMDDAVIFPLDMARVDGRQNIQKLWQGLMDMGVSEPKFTTLNVQESGDFAFESGIFSLKGPGKDSKRVDIAGIYVEVWRKGQDGGWKLYRTIANLDPAK

SEQ ID NO: 190 - protein, 128 aa, Artificial Sequence
  Synthetic
GQSAKEAIEAALADFVKAYNGKDAAGVGSKYMDDAAIFPLDMARVDGRQNIQKLWQGLMDTGVSEPKFTTLVVQESGDFAFESGSFSLKGPGPDSKLVDIAGKYVEVWRKGQDGGWKLYRTIANLDPA

SEQ ID NO: 191 - protein, 129 aa, Artificial Sequence
  Synthetic
GQSAKEAIEAALADFVKAYNSKDAAGVASKYMDDAAIFPLDMAPVDGRQNIQKLWQGLMDMGVSEPKFTTLNVQESGDFAFESGSFSLKGPSKDSKLVDIAGIYVEVWRKGQDGGWKLYRTIANLDPAK

SEQ ID NO: 192 - protein, 129 aa, Artificial Sequence
  Synthetic
GHSAKEAIEAALADFVKAYNSKDAAGVASKYMDDAAIFPLDMAPVDGRQNIQKLWQGLMDLGVSEPKFTTLNVQESGDFAFESGGFSLKGPGKDSKLVDIAGIYVEVWRKGLDGGWKLYRTIANLDPAK

SEQ ID NO: 193 - protein, 129 aa, Artificial Sequence
  Synthetic
GQSAKEAIEAVLADFVKAYNSKDAAGVASKYMDDAAIFPLDMAPVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 194 - protein, 129 aa, Artificial Sequence
  Synthetic
DQSAKEAIEAALADFVKVYNSKNAAGVASKYMDDAVIFPLDMARVDGRQNIQKLWQGLMDMGVSEPKFTTLNVQESGDFAFESGSFSLKGPGKDSKLVDIAGIYVEVWRKGQDGGWKLYRTIASLDPAK

SEQ ID NO: 195 - protein, 129 aa, Artificial Sequence
  Synthetic
GQSAKEAIEAALADFVKVYNSKDVAGVASKYMDDAVIFPLDMARVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 196 - protein, 129 aa, Artificial Sequence
  Synthetic
GQSAKEAIEAALADFVKAYNSKDAAGVASKYMDDPAIFPLDMAPVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 197 - protein, 129 aa, Artificial Sequence
  Synthetic
GQSAKEAIEAALADFVKVYNSKDAAGVASKYMDDAAIFPLDMAPVDGRQNIQKLWQGLMDMGVSEPKFTTLNVQKSGDFAFESGSFSLKGPGKDSKLVDIAGIYVEVWRKGQDGGWKLYRTIANLDPAK

SEQ ID NO: 198 - protein, 129 aa, Artificial Sequence
  Synthetic
GQSGKEAIEAALADFVKAYNGKDAAGVASKYMDDAAIFPLDMARVDGRQNIQKLWQGLMDTGVSEPKFTTLVVQESGDFAFESGSFSLKGPGPDSRLVDIAGKYVEVWRKGQDGGWKLYRTIANLDPAK

SEQ ID NO: 199 - protein, 129 aa, Artificial Sequence
  Synthetic
GQSAKEAIEAALADFVKAYNSKDAAGVANKYMDDAAIFPLDMAPVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 200 - protein, 129 aa, Artificial Sequence
  Synthetic
GQSAKEAIEAALADFVKAYNGKDAAGVASKYMDDAAIFPLDMARVDGRQNIQKLWQGLMDTGVSEPKFTTLVVQESGDFAFESGSFSLKGPGPDSKLVDIAGKYVEVWRKGQDGGWKLYRTIANLDPAK

SEQ ID NO: 201 - protein, 129 aa, Artificial Sequence
  Synthetic
GQSAKEAIEAALADFVKAYNSKDAAGVASKYMDDAAIFPLDMAPVDGRQNIQKLWQGLMDMGVSELKLTTLDVQESGDFAFESGSFSLKGPGKDSKLVDIAGKYVEVWRKGQDGGWKLYRTIANLDPAK

SEQ ID NO: 202 - protein, 129 aa, Artificial Sequence
  Synthetic
GQSAKEAIEAALADFVKAYNSKDAAGVASKYMDDAAIFPLDMAPVDGRQNIQKLWQGLMDMGVSEPKFTTLDVQESGDFAFESGSFSLKGPGKDSKLVDIAGKYVEVWRKGQDGGWKLYRTIANLDPAK

SEQ ID NO: 203 - protein, 129 aa, Artificial Sequence
  Synthetic
GQSAKEAIEAALADFVKAYNSKDAAGVASKYMDDAAIFPLDMAPVDGRQNILKLWQGLMDLGVSGPKFTTLVVQESGDFAFESGSFSLKGPGKDSKLVDIAGKYVEVWRKGQDGGWKLYRTIANLDTAK

SEQ ID NO: 204 - protein, 129 aa, Artificial Sequence
  Synthetic
GQSAKEAIEAALADFVKAYNSKDVAGVASKYMDDAVIFPLDMAPVDGRQNIQKLWQGLMDMGVSGVKLTTLDVQESGDFAFESGSFSLKGPGKDSKLVDIAGKYVEVWRKGQDGGWKLYRTIANLDPAK

SEQ ID NO: 205 - protein, 129 aa, Artificial Sequence
  Synthetic
GQSAKGAIEAALADFVKAYNSKDAAGVASKYMDDAAIFPLDMAPVDGRQNIQKLWLGLMDMGVSEPKLTTLDVQESGDFAFESGSFSLKGPGKDSKLVDIAGKYVEVWRKGQDGGWKLYRTIANLDPAK

SEQ ID NO: 206 - protein, 129 aa, Artificial Sequence; identical to SEQ ID NO: 21
  Synthetic

SEQ ID NO: 207 - protein, 129 aa, Artificial Sequence
  Synthetic
GQSSKEAIEVALADFVKAYNSKDAAGVASKYMDDAAIFPLDMARVDGRQNIQKLWQGLMDMGVSEPKLTTLDVQESGDFAFESGSFSLKGPSKDSKLVDIAGKYVEVWRKGPDGGWKLYRTIANLDPAK

SEQ ID NO: 208 - protein, 129 aa, Artificial Sequence
  Synthetic
GQSAKEAIEAALADFVKAYNSKDAAGVASKYMDDAAIFPLDMAPVDGRQNIQELWQGLMDMGVSELKLTTLDVQESGDFAFESGNFSLKGPGKDSKLVDIAGKYVEVWRKGQDGGWKLYRTIANLDPAK

SEQ ID NO: 209 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLDVQESGDFAFESGSFCLKGPGKDSKLVDIAGKYVEVWRKGQDGGWKLYRTIANLDPAK

SEQ ID NO: 210 - protein, 129 aa, Artificial Sequence
  Synthetic
GQSAKEAIEAALADFVKAYNSKDAAGVASKYMDDAAIFPLEMAPVDGRQNIQKLWQGLMDMGVSEPKLTTLDVQESGDFAFESGSFSLKGPGKDSKLVDIAGKYVEVWRKGQDGGWKLYRTIANLDPAK

SEQ ID NO: 211 - protein, 129 aa, Artificial Sequence
  Synthetic
GQSAKEAIEAALADFVKAYNSKDAAGVASKYMDDATIFPLDMARVDGRQNIQKLWQGLMDMGVSELKSTTLDVQESGDFAFESGSFSLKGPGKDSKLVDVAGKYVVVWRKGQDGGWKLYRTISNLDPAK

SEQ ID NO: 212 - protein, 129 aa, Artificial Sequence
  Synthetic
GQSAKEAIEAALADFVKAYNSKDAAGVASKYMDDAAIFPLDMARVDGRQNIQKLWQGLMDMGVSELKSTTLDVQESGDFAFESGSFSLRGPGKDSKLVDVAGKYVVVWRKGQDGGWKLYRTISNLDPAK

SEQ ID NO: 213 - protein, 129 aa, Artificial Sequence; identical to SEQ ID NO: 25
  Synthetic

SEQ ID NO: 214 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKLTTLGVQESGDFAFESGSFSLKGPGKDNKLVDIAGKYVEVWRKGQDGGWKLYRTIANLDPAK

SEQ ID NO: 215 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKFTTLNVQESGDFAFESGSFSLKGPGKDSKLVDIAGIYVEVWRKGQDGGWKLYRTIANLDPAK

SEQ ID NO: 216 - protein, 129 aa, Artificial Sequence
  Synthetic
GQSAKVAIEAALADFVKVYNSKDAAGVASKYMDDAAIFPLDMARVDGRQNIQKLWQGLMDMGVSEPKFTTLNVQESGDFAFESGSFSLKGPGKDSKLVDIAGNYVEVWRKGQGGGWKLYRTIANLDPAK

SEQ ID NO: 217 - protein, 129 aa, Artificial Sequence
  Synthetic
GQSAKEAIEAALADFVKAYNSKDAAGVASKYMDDAAIFPLDMAPVDGRQNIQKLWQGLMDMGVSGVKLTTLDVQESGDFAFESGSFSLKGPGKDSKLVDIAGKYVEVWRKGQDGGWKLYRTIANLDPAN

SEQ ID NO: 218 - protein, 129 aa, Artificial Sequence
  Synthetic
GQSAKVAIEAALADFVKVYNSKDAAGVASKYMDDAAIFPLDMAPVDGRQNIQKLWQGLMDMGVSEPKFTTLNVQESGDFAFESGSFSLKGPGKDSKLVDIAGNYVEVWRKGQGGGWKLYRTIANLDPAK

SEQ ID NO: 219 - protein, 129 aa, Artificial Sequence
  Synthetic
GQSAKEAIEAALADFVKAYNSKDAAGVASKYMDDAAIFPLDMARVDGRQNIQKLWQGLMDMGVSEPKFTTLNVQESGDFAFESGSFSLKGPGKDSKLVDIAGNYVEVWRKGQDGGWKLYRTIANLDPAK

SEQ ID NO: 220 - protein, 127 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDIAGKYVEVWRKADPPPSSFGTRFMVPYN

SEQ ID NO: 221 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVRESGDFAFESGSFSLKGPGKDSKLVDIAGKYVEVWRKGQDGGWKLYRTISNLDPAK

SEQ ID NO: 222 - protein, 129 aa, Artificial Sequence
  Synthetic
GQSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDVAGKYVEVWRKGQDGGWKLYRTIANLDPAK

SEQ ID NO: 223 - protein, 129 aa, Artificial Sequence; identical to SEQ ID NO: 5
  Synthetic

SEQ ID NO: 224 - protein, 129 aa, Artificial Sequence
  Synthetic
GQSAKEAIEAALADFVKAYNSKDAAGVASKYMDDAAIFPLDMARVDGRQNTQKLWQGLMDMGVSELKLTTLDVQESGDFAFESGSFSLKGPGKDSKLVDIAGKYVEVWRKGQDGGWKLYRVIVNLDPAK

SEQ ID NO: 225 - protein, 129 aa, Artificial Sequence
  Synthetic
GQSAKGAIEAALADFVKAYNSKDAAGVASKYMDDAAIFPLDMARVDGRQNIQKLWQGLMDMGVSELKLTTLDVQESGDFAFESGSFSLKGPGKDSKLVDIAGKYVEVWRKGQDGGWKLYRVIVNLDPAK

SEQ ID NO: 226 - protein, 129 aa, Artificial Sequence; identical to SEQ ID NO: 17
  Synthetic

SEQ ID NO: 227 - protein, 129 aa, Artificial Sequence; identical to SEQ ID NO: 16
  Synthetic

SEQ ID NO: 228 - protein, 129 aa, Artificial Sequence
  Synthetic
ARSAKEAIEAALADFVKAYNSKDAAGVASKYMDDAAIFPLDMARVDGRQNIQKLWQGLMDMGVSELKLTTLDVQESGDFAFESGSFSLKGPGKDSKLVDIAGKYVEVWRKGQDGGWKLYRAIANLDPAK

SEQ ID NO: 229 - protein, 129 aa, Artificial Sequence
  Synthetic
GQSAKEAIEAALADFVKAYNSKDAAGVASKYMDDAAIFPLDMARVDGRQNIQKLWQGLMDMGVSEVKLTTLDVQGSGDFAFESGSFSAKGPGKDSKLVDMAGKYVEVWRKGQDGGWKLYRVIANLDPAK

SEQ ID NO: 230 - protein, 129 aa, Artificial Sequence
  Synthetic
GQSAKEAIEAALADFVKAYNSKDAAGVACKYMDDAAIFPLDMARVDGRQNIQKLWQGLMDMGVSEVKLTTLDVQESGDFAFESGSFSAKGPGKDSKLVDMAGKYVEVWRKGQDGGWKLYRVIANLDPAK

SEQ ID NO: 231 - protein, 148 aa, Artificial Sequence
  Synthetic
SGREQGHMNAKEILVHALRLVENGDARGFCDLFHPEGVMEFPYAPPGYKTRFEGRETIWAHMRLFPEHLTIRFTDVQFYETADPDLAIGEFHGDGVATVSGGKLAQDFISVLRTRDGQILLSRIFWNPLRHLEALGGVEAAAKIVQGA

SEQ ID NO: 232 - protein, 136 aa, Artificial Sequence
  Synthetic
NLYFQGHMNAKEILVHALRLVENGDARGFCDLFHPEGVMEFPYAPSGYKTRFEGAETIWAHMRLFPEPLTIRFTDVQFYETADPDLAIGEFHGDGVATVSGGKLAQDFISVLRTRDGQILLSRIFWNPLRHLEALV

(19) United States
(12) Patent Application Publication
(10) Pub. No.: US 2017/0322229 A1
(43) Pub. Date: Nov. 9, 2017

(54) HIGH AFFINITY VITAMIN D3 BINDING PROTEINS

(71) Applicant: UNIVERSITY OF WASHINGTON, Seattle, WA (US)

(72) Inventors: David BAKER, Seattle, WA (US); Austin DAY, Seattle, WA (US); Per Junior GREISEN, Seattle, WA (US)

(21) Appl. No.: 15/541,102

(22) PCT Filed: Feb. 2, 2016
(86) PCT No.: PCT/US16/16054

§ 371 Date: Jun. 30, 2017

Related U.S. Application Data
(60) Provisional application No. 62/110,710, filed on Feb. 2, 2015.

(51) Int. Cl. G01N 33/82; C07K 14/00

(52) U.S. Cl. CPC G01N 33/82; C07K 14/00; G01N 2800/104; G01N 2800/285; G01N 2800/2842

(57) ABSTRACT

The present disclosure provides isolated polypeptides with vitamin D3 binding activity and methods for their use as detection agents. In another aspect, the invention provides recombinant expression vector comprising an isolated nucleic acid of the invention operably linked to a control sequence. In another aspect, the invention provides recombinant host cells comprising the recombinant expression vector of the invention. In another aspect, the invention provides methods for detecting vitamin D3 or one of its metabolites, such as 25-D3, comprising contacting a sample of interest with a detectable polypeptide of the invention.

### CROSS-REFERENCE

This application claims priority to U.S. Provisional Patent Application Ser. No. 62/110,710 filed Feb. 2, 2015, incorporated by reference herein in its entirety.

### STATEMENT REGARDING FEDERALLY SPONSORED RESEARCH

This invention was made with U.S. government support under HDTRA1-10-1-0040, awarded by the Defense Threat Reduction Agency. The U.S. Government has certain rights in the invention.

### BACKGROUND

Cholecalciferol, also known as toxiferol, is a form of vitamin D, also called vitamin D3. It is structurally similar to steroids such as testosterone, cholesterol, and cortisol. Vitamin D metabolites have been identified as potential clinical markers for autoimmune and chronic diseases such as multiple scelerosis, lupus, and fibromyalgia. In particular, 25-Hydroxycholecalciferol (25-D3), the hormonally active variant form of Vitamin D3 is clinically relevant and of interest for several indications. There is presently an unmet need for assays that detect and molecules and devices that specifically bind to vitamin D3 and its metabolites.

### SUMMARY OF THE INVENTION

In a first aspect, the invention provides isolated polypeptides comprising a polypeptide at least 70% identical over the full length of the amino acid sequence of SEQ ID NO:1. In other embodiments, the polypeptide is at least 80% or 90% identical over the full length of the amino acid sequence of SEQ ID NO:1. In other embodiments, the polypeptide comprises the amino acid sequence of SEQ ID NO:2 or SEQ ID NO:3. In various further embodiment, the polypeptide comprises the amino acid sequence of a peptide selected from the group consisting of SEQ ID NOS: 1-230. In another aspect, the invention provides isolated polypeptides comprising the amino acid sequence of SEQ ID NO: 231 or 232.

In one embodiment, the polypeptides of the invention may comprise a detectable tag.

In another aspect, the invention provides isolated nucleic acids encoding the polypeptide of any embodiment of the invention. In another aspect, the invention provides recombinant expression vector comprising an isolated nucleic acid of the invention operably linked to a control sequence. In another aspect, the invention provides recombinant host cells comprising the recombinant expression vector of the invention.

In another aspect, the invention provides methods for detecting vitamin D3 or one of its metabolites, comprising:

(a) contacting a sample of interest with a polypeptide according to any one of claims 1-9 under suitable conditions for binding the polypeptide to vitamin D3 or one of its metabolites present in the sample to form a polypeptide-vitamin D3 (or one of its metabolites) binding complex, and

(b) detecting the binding complex.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1: A) Fluorescence polarization data for 25-D3 binder CDL2, showing an approximate Kd of 2.1 uM. B) Yeast surface display and flow cytometry titration for evolved variant CDL2.1. Approximate Kd values are 319 nM (black) for 25-D3 and 1.9 uM for vitamin D3 (red). C) Fluorescence polarization data for 25-D3 binder CDL2.2. The approximate Kd value is 188 nM. D) A structure comparison between CDL2 and CDL2.1 highlighting mutations introduced into the binding pocket during evolution.

FIG. 2: A) An alignment between the crystal structure of CDL2.1 and the original model CDL2 with 25-D3 docked in. The RMSD is 1.066° A. B) Crystal structure of CDL2.1 demonstrating the presence of water in the hydrogen bonding interaction. C) Surface representation of CDL2. D) Surface representation of the crystal structure of CDL2.1.

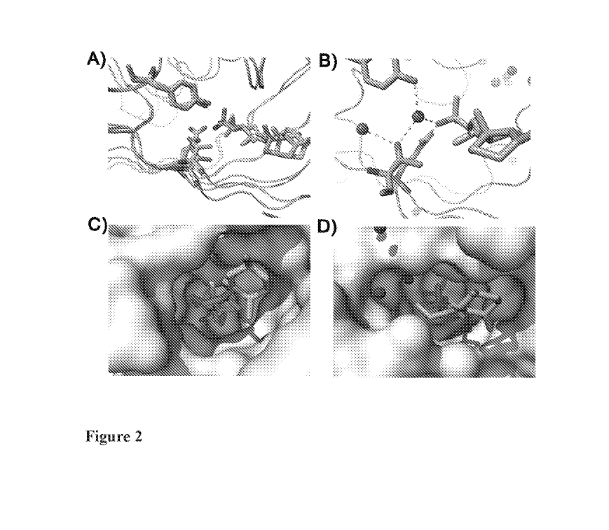

FIG. 3: Rosetta docking plot of 25-D3 docked into several structures. A) Docking plot for the original design CDL2. B) Docking plot for a model variant that contains the evolved mutations of CDL2.1 in the backbone structure of CDL2. C) Docking plot for crystal structure of variant CDL2.1. For all plots, the y-axis represents the Rosetta interface energy and the x-axis represents the root mean squared deviation of the final positions of each docking trajectory to the ligand position in the CDL2.1 crystal structure.

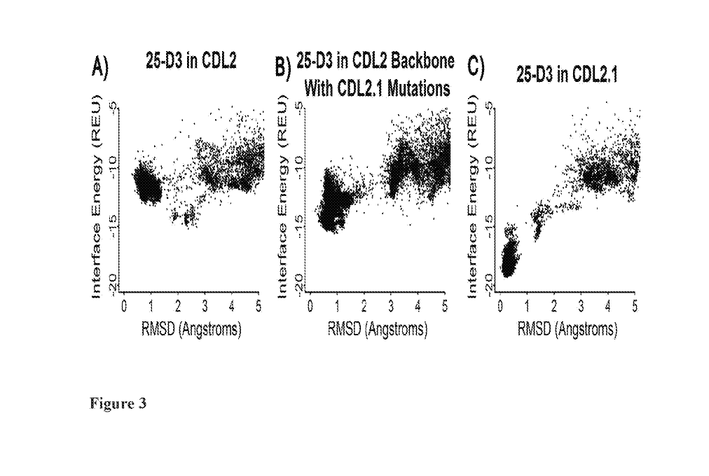

### DETAILED DESCRIPTION

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

The terms “a,” “an,” “the” and similar referents used in the context of describing the invention (especially in the context of the following claims) are to be construed to cover both the singular and the plural, unless otherwise indicated herein or clearly contradicted by context.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used throughout the present application, the term “polypeptide” is used in its broadest sense to refer to a sequence of subunit amino acids. The polypeptides of the invention may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo. such as by PEGylation, HESylation, PASylation, glycosylation, etc. Such linkage can be covalent or non-covalent as is understood by those of skill in the art.

In a first aspect, the invention provides isolated polypeptides comprising or consisting of a polypeptide at least 70% identical over the full length of the amino acid sequence of SEQ ID NO:1 (see Table 1)

TABLE 1
SEQ ID NO: 1

| Residues | AAs |
|---|---|
| 1 | M, or absent |
| 2 | S, A, D, G, L, or absent |
| 3 | H, Q, R, P, K |
| 4 | S, T, N, R, I |
| 5 | S, A, G, V |
| 6 | H, Q, K, E |
| 7 | G, E, V |
| 8 | A, T, P, V |
| 9 | I, V |
| 10 | K, E |
| 11 | S, A, V |
| 12 | A, T, V |
| 13 | L |
| 14 | A |
| 15 | D, E |
| 16 | S, F, Y, L |
| 17 | A, L, V |
| 18 | K |
| 19 | S, A, G, V |
| 20 | F, C, Y |
| 21 | N, K |
| 22 | S, N, C, R, P, G |
| 23 | M, N, K |
| 24 | N, D |
| 25 | A, T, G, V |
| 26 | A, T |
| 27 | D, G |
| 28 | L, V |
| 29 | A, V |
| 30 | S, C, N, R, G |
| 31 | N, K |
| 32 | S, Y |
| 33 | T, M, K, I, L, V |
| 34 | N, D |
| 35 | D, G |
| 36 | A, P, V |
| 37 | S, A, T, P, E, V |
| 38 | I |
| 39 | F, Y |
| 40 | P, L |
| 41 | Q, M, P, L |
| 42 | D, G, E |
| 43 | M |
| 44 | A, T, V |
| 45 | H, S, P, R, L, |
| 46 | A, V |
| 47 | D, G, V |
| 48 | G |
| 49 | C, P, R |
| 50 | Q, R |
| 51 | D, N, Y |
| 52 | S, T, I |
| 53 | Q, P, E, L |
| 54 | R, K, E |
| 55 | M, L |
| 56 | W, L |
| 57 | Q, L |
| 58 | D, G |
| 59 | Q, L |
| 60 | T, M, K, I, L |
| 61 | D |
| 62 | T, M, L |
| 63 | C, G |
| 64 | M, V |
| 65 | S, N, C |
| 66 | D, G, E |
| 67 | P, L, V |
| 68 | K, E |
| 69 | S, F, L |
| 70 | T |
| 71 | S, A, T, P, I |
| 72 | Q, M, L |
| 73 | N, D, G, V |
| 74 | V |
| 75 | Q, R |
| 76 | K, G, E |
| 77 | S, C, N |
| 78 | G |
| 79 | D, Y, V |
| 80 | F, I, V |
| 81 | A, T, P, V |
| 82 | S, F, Y |
| 83 | E, V |
| 84 | S, G |
| 85 | G |
| 86 | S, N, I, R, G |
| 87 | F, I, L |
| 88 | S, C, R |
| 89 | A, P, L, V |
| 90 | R, K |
| 91 | S, G |
| 92 | S, P |
| 93 | S, D, G, V |
| 94 | Q, T, N, R, P, K |
| 95 | D |
| 96 | S, C, N, I, G |
| 97 | R, K, E |
| 98 | M, R, L |
| 99 | A, V |
| 100 | D, G |
| 101 | M, N, I, V |
| 102 | A, T, V |
| 103 | C, G |
| 104 | N, I, K, E |
| 105 | F, Y |
| 106 | E, V |
| 107 | M, K, G, E, V |
| 108 | V |
| 109 | W |
| 110 | R, G |
| 111 | N, K |
| 112 | A, G |
| 113 | Q, D, P, R, K, L |
| 114 | N, D, P, G, Y |
| 115 | P, G |
| 116 | S, D, P, G |
| 117 | S, W, R, L |
| 118 | S, T, K |
| 119 | F, L |
| 120 | Y |
| 121 | H, C, R, G |
| 122 | S, A, T, I, V |
| 123 | T, R, I |
| 124 | F, A, S, T, V |
| 125 | S, N |
| 126 | Q, M, P, L |
| 127 | N, D, G, E, V |
| 128 | T, P, L |
| 129 | S, A, T, Y, V, or absent |
| 130 | N, R, K, E, or absent |

The polypeptides of all aspects/embodiments of the invention bind to D3 and to 25-Hdroxycholecalciferol (25-D3) and can thus be used, for example, in the context of biosensors for specific quantification of vitamin D3 and 25-D3. The polypeptides of the invention provide a cheaper, selective alternative to currently used antibodies. The polypeptides of the invention are at least 70% identical with to the amino acid sequence of SEQ ID NO:1 over its full length. In various embodiments, the polypeptides of the invention are at least 75%, 80%, 85%, 90%, 91%, 92%, 93%, 94%, 95%, 96%, 97%, 98%, or 99% identical with to the amino acid sequence of SEQ ID NO:1 over its full length.

In one embodiment, the isolated peptides comprising or consisting of the amino acid sequence in SEQ ID NO:2 (see Table 2).

TABLE 2
SEQ ID NO: 2

| Residue | AAs |
|---|---|
| 1 | M or is absent |
| 2 | D, G, L, or is absent |
| 3 | Q, P |
| 4 | S, T |
| 5 | A |
| 6 | H, K |
| 7 | E |
| 8 | A |
| 9 | I |
| 10 | E |
| 11 | A |
| 12 | A |
| 13 | L |
| 14 | A |
| 15 | D |
| 16 | F |
| 17 | L, V |
| 18 | K |
| 19 | A, V |
| 20 | F, Y |
| 21 | N |
| 22 | S, G |
| 23 | K |
| 24 | D |
| 25 | A |
| 26 | A |
| 27 | D, G |
| 28 | V |
| 29 | A |
| 30 | S |
| 31 | K |
| 32 | Y |
| 33 | M |
| 34 | D |
| 35 | D, G |
| 36 | A |
| 37 | A, V |
| 38 | I |
| 39 | F |
| 40 | P |
| 41 | L |
| 42 | D |
| 43 | M |
| 44 | A |
| 45 | R, P |
| 46 | V |
| 47 | D |
| 48 | G |
| 49 | R |
| 50 | Q |
| 51 | N, Y |
| 52 | S, I |
| 53 | Q |
| 54 | R, K |
| 55 | L |
| 56 | W |
| 57 | Q |
| 58 | G |
| 59 | L |
| 60 | M, I |
| 61 | D |
| 62 | T, M |
| 63 | G |
| 64 | V |
| 65 | S |
| 66 | G, E |
| 67 | P, L |
| 68 | K |
| 69 | F, L |
| 70 | T |
| 71 | T, I |
| 72 | M, L |
| 73 | D, N, V |
| 74 | V |
| 75 | Q |
| 76 | K, E |
| 77 | S |
| 78 | G |
| 79 | D |
| 80 | F |
| 81 | A |
| 82 | F,Y |
| 83 | E |
| 84 | S |
| 85 | G |
| 86 | S, R |
| 87 | F |
| 88 | S |
| 89 | L |
| 90 | K |
| 91 | G |
| 92 | P |
| 93 | D, G |
| 94 | P, K |
| 95 | D |
| 96 | S |
| 97 | K |
| 98 | L |
| 99 | V |
| 100 | D, G |
| 101 | I, |
| 102 | A |
| 103 | G |
| 104 | I, K |
| 105 | Y |
| 106 | V |
| 107 | E |
| 108 | V |
| 109 | W |
| 110 | R |
| 111 | K |
| 112 | G |
| 113 | Q |
| 114 | D, G |
| 115 | G |
| 116 | G |
| 117 | W |
| 118 | K |
| 119 | L |
| 120 | Y |
| 121 | H, R |
| 122 | T |
| 123 | I |
| 124 | A |
| 125 | N |
| 126 | L |
| 127 | D, G |
| 128 | P |
| 129 | A, or is absent |
| 1301 | R, K, or is absent |

In another embodiment, the isolated polypeptides comprising or consisting of the amino acid sequence of SEQ ID NO:3 (see Table 3).

TABLE 3
SEQ ID NO: 3

| Residue | AAs |
|---|---|
| 1 | M, or is absent |
| 2 | D, G, L, or is absent |
| 3 | Q, P |
| 4 | S, T |
| 5 | A |
| 6 | H, K |
| 7 | E |
| 8 | A |
| 9 | I |
| 10 | E |
| 11 | A |
| 12 | A |
| 13 | L |
| 14 | A |
| 15 | D |
| 16 | F |
| 17 | V |
| 18 | K |
| 19 | A, V |
| 20 | Y |
| 21 | N |
| 22 | S |
| 23 | K |
| 24 | D |
| 25 | A |
| 26 | A |
| 27 | G |
| 28 | V |
| 29 | A |
| 30 | S |
| 31 | K |
| 32 | Y |
| 33 | M |
| 34 | D |
| 35 | D |
| 36 | A |
| 37 | A, V |
| 38 | I |
| 39 | F |
| 40 | P |
| 41 | L |
| 42 | D |
| 43 | M |
| 44 | A |
| 45 | R, P |
| 46 | V |
| 47 | D |
| 48 | G |
| 49 | R |
| 50 | Q |
| 51 | N |
| 52 | I |
| 53 | Q |
| 54 | K |
| 55 | L |
| 56 | W |
| 57 | Q |
| 58 | G |
| 59 | L |
| 60 | M |
| 61 | D |
| 62 | M |
| 63 | G |
| 64 | V |
| 65 | S |
| 66 | E |
| 67 | P |
| 68 | K |
| 69 | F |
| 70 | T |
| 71 | T |
| 72 | L |
| 73 | N |
| 74 | V |
| 75 | Q |
| 76 | K, E |
| 77 | S |
| 78 | G |
| 79 | D |
| 80 | F |
| 81 | A |
| 82 | F |
| 83 | E |
| 84 | S |
| 85 | G |
| 86 | S |
| 87 | F |
| 88 | S |
| 89 | L |
| 90 | K |
| 91 | G |
| 92 | P |
| 93 | G |
| 94 | K |
| 95 | D |
| 96 | S |
| 97 | K |
| 98 | L |
| 99 | V |
| 100 | D, G |
| 101 | I |
| 102 | A |
| 103 | G |
| 104 | I |
| 105 | Y |
| 106 | V |
| 107 | E |
| 108 | V |
| 109 | W |
| 110 | R |
| 111 | K |
| 112 | G |
| 113 | Q |
| 114 | D |
| 115 | G |
| 116 | G |
| 117 | W |
| 118 | K |
| 119 | L |
| 120 | Y |
| 121 | R |
| 122 | T |
| 123 | I |
| 124 | A |
| 125 | N |
| 126 | L |
| 127 | D, G |
| 128 | P |
| 129 | A, or is absent |
| 130 | R, K, or is absent |

Polypeptides within the scope of SEQ ID NOS:2-3 show particularly strong binding to and selectivity for 25-D3 as shown via yeast surface display.

In various further embodiments, the isolated polypeptides comprises or consists of a peptide with an amino acid sequence selected from the group consisting of the following, each of which is believed to bind to 25-D3 and/or D3 generated via homology, related proteins, or sequences obtained from library sorting that showed a signal on yeast:

4424 (CDL2):
(SEQ ID NO: 4)
GQSAKEAIEAALADFVKAYNSKDAAGVASKYMDDAAIFPLDMARVDGRQN
IQKLWQGLMDMGVSELKLTTLDVQESGDFAFESGSFSLKGPGKDSKLVDV
AGKYVVVWRKGQDGGWKLYRTISNLDPAK
4424 + 106E:
(SEQ ID NO: 5)
GQSAKEAIEAALADFVKAYNSKDAAGVASKYMDDAAIFPLDMARVDGRQN
IQKLWQGLMDMGVSELKLTTLDVQESGDFAFESGSFSLKGPGKDSKLVDV
AGKYVEVWRKGQDGGWKLYRTISNLDPAK
4424 + 106E Error Prone Neg Sort Mutant:
(SEQ ID NO: 6)
GQSAKEIEAALADEVKAYNSKDAAGVASKYMDDAAIFPLDMARVDGRQNI
QKLWQGLMDMGVSELKLTTLDVQESGDIAFESGSFSLKGPGKDSKLVDVA
GKYVEVWRKGQDGGWKLYRTISNLDPAK
4424 + 106E Error Prone Neg Sort Mutant:
(SEQ ID NO: 7)
GQIAKEAIEAALADFVKAYNSKDAAGVASKYMDDAAIFPLDMARVDGRQN
IQKLWQGLMDMGMSELKLTTLDVQESGDFAFESGSFSLKGPGKDSKLVDV
AGKYVEVWRKGQDGGWKLYRTISNPDPAK
4424 + 106E Error Prone Neg Sort Mutant:
(SEQ ID NO: 8)
GQSAKEAIEAVLADFVKAYNSKDAAGVVSKYMNDAAIFPLDMARVDGRQN
IQKLWQGLMDMGVSELKLTTLDVQESGDFAFESGSFSLKGPGKDSKLVDV
AGKYVEVWRKGQDGGWKLYCTISNLDPAK
4424 + 106E Error Prone Neg Sort Mutant:
(SEQ ID NO: 9)
GQSAKEAIEAALADFVKAYNSKDAAGVASKYMDDAAIFPLDMARVDGRQN
IQKLWQGLMDMGVSELKLTTLDVQESGDFAFESGSFSLKGPGKDSKLVDV
AGKYVVVWRKGQDGGWKLYRTISNLDPAK
4424 + 106E Error Prone Neg Sort Mutant:
(SEQ ID NO: 10)
GQSAQEAIEAALADFVKAYNSKDAAGVASKYMDDAAIFPLDMARVDGRQN
IQKLWQGLMDMGVSELKLTTLDVQESGDFAFESGSFSLKGPGKDSKLVDV
AGKYVEVWRKGQDGGWKLYRTISNLDPAK
4424 + 106E Error Prone Neg., Sort Mutant:
(SEQ ID NO: 11)
GQSAKEAIEAALADFVKAYNSKDAAGVASKYMDDAAIFPLDMARVDGRQN
IQKLWQGLMDMGVSELKLTTLDVQESGDFAFESGSFSLKGPGKDSKLVDV
AGKYVEVWRKGQDGDWKLYRTISNLDLAK
4424 + 106E Error Prone Neg Sort Mutant:
(SEQ ID NO: 12)
GQSAKEAIEAALADFVKAYNSKDAAGVASKYIMDDAAIFPLDMARVDGRQ
DIQKLWQGLMDMGVSELKLTTLDVQESGDFAFESGSFSLKGPGKDSKLVD
VAGKYVEVWRKGQDGGWKLYRTISNLDPAK
4424 + 106E Error Prone Neg Sort Mutant:
(SEQ ID NO: 13)
GQSAKEAIEAALADFVKAYNSKDAAGVASKYMDDAAIFPLDMARVDGRQN
IQKLWQGLMDMGVSELKLTTLDVQESGDFAFESGSFSLKGPGKDSKLVDV
AGKYVEVWRKGQDGGWKLYRTISNLNPAK
Model 4 (4424 + V106E + V100I + S123A):
(SEQ ID NO: 14)
GQSAKEAIEAALADFVKAYNSKDAAGVASKYMDDAAIFPLDMARVDGRQN
IQKLWQGLMDMGVSELKLTTLDVQESGDFAFESGSFSLKGPGKDSKLVDI
AGKYVEVWRKGQDGGWKLYRTIANLDPAK
Model 1 (V106E + T121V + S123A + V100M):
(SEQ ID NO: 15)
GQSAKEAIEAALADFVKAYNSKDAAGVASKYMDDAAIFPLDMARVDGRQN
IQKLWQGLMDMGVSEVKLTTLDVQESGDFAFESGSFSAKGPGKDSKLVDM
AGKYVEVWRKGQDGGWKLYRVIANLDPAK
Model 2 (V106E + S123A + T121A + V100I):
(SEQ ID NO: 16)
GQSAKEAIEAALADFVKAYNSKDAAGVASKYMDDAAIFPLDMARVDGRQN
IQKLWQGLMDMGVSELKLTTLDVQESGDFAFESGSFSLKGPGKDSKLVDI
AGKYVEVWRKGQDGGWKLYRAIANLDPAK
Model 3 (V106E + S123V + T121V + V100I):
(SEQ ID NO: 17)
GQSAKEAIEAALADFVKAYNSKDAAGVASKYMDDAAIFPLDMARVDGRQN
IQKLWQGLMDMGVSELKLTTLDVQESGDFAFESGSFSLKGPGKDSKLVDI
AGKYVEVWRKGQDGGWKLYRVIVNLDPAK
M26 (M4 + A36P + L66P + A80P):
(SEQ ID NO: 18)
GQSAKEAIEAALADFVKAYNSKDAAGVASKYMDDAPIFPLDMARVDGRQN
IQKLWQGLMDMGVSEPKLTTLDVQESGDFPFESGSFSLKGPGKDSKLVDI
AGKYVEVWRKGQDGGWKLYRTIANLDPAK
M30 (M4 + R44P + E65G +L 66V):
(SEQ ID NO: 19)
GQSAKEAIEAALADFVKAYNSKDAAGVASKYMDDAAIFPLDMAPVDGRQN
IQKLWQGLMDMGVSGVKLTTLDVQESGDFAFESGSFSLKGPGKDSKLVDI
AGKYVEVWRKGQDGGWKLYRTIANLDPAK
M16 (M4 + Q2K + L66P):
(SEQ ID NO: 20)
GKSAKEAIEAALADFVKAYNSKDAAGVASKYMDDAAIFPLDMARVDGRQN
IQKLWQGLMDMGVSEPKLTTLDVQESGDFAFESGSFSLKGPGKDSKLVDI
AGKYVEVWRKGQDGGWKLYRTIANLDPAK
B5 (4424 + 106E + L66P + Q49R):
(SEQ ID NO: 21)
GQSAKEAIEAALADFVKAYNSKDAAGVASKYMDDAAIFPLDMARVDGRQN
IQKLWQGLMDMGVSEPKLTTLDVQESGDFAFESGSFSLKGPGKDSKLVDI
AGKYVEVWRKGQDGGWKLYRTIANLDPAK
M6:
(SEQ ID NO: 22)
GQSAKEAIEAAILADFVKAYNSKDAAGVASKYMDDAAIFPLDMARVDGRQ
NIQKLWQGLMDMGVSEPKLTTLDVQESGDFAFESGSFSLKGPGKDSKLVD
IAGKYVEVWRKGQDGGWKLYRTIANLDPAK
M23:
(SEQ ID NO: 23)
GQSAKEAIEAALADFVKAYNSKDAAGVASKYMDDAAIFPLDMARVDGRQ
NILKLWQGLMDMGVCELKFTTLDVQESGDFAFESGSFSLKGPGKDSKLV
DIAGKYVEVWRKGQDGGWKLYRTIANLDPAK
H34 (M30 + A18V + D72N + K103I):
(SEQ ID NO: 24)
GQSAKEAIEAALADFVKVYNSKDAAGVASKYMDDAAIFPLDMAPVDGRQ
NIQKLWQGLMDMGVSGVKLTTLNVQESGDFAFESGSFSLKGPGKDSKLV
DIAGIYVEVWRKGQDGGWKLYRTIANLDPAK
F4:
(SEQ ID NO: 25)
GQSAKEAIEAALADFVKAYNSKDAAGVASKYMDDAAIFPLDMARVDGRQ
NIQKLWQGLMDMGVSEPKLTTLDVQESGDFAFESGSFSLKGPGKDIKLV
DIAGKYVEVWRKGQDGGWKLYRTIANLDPAK
F14:
(SEQ ID NO: 26)
GQSAKEAIEAALADFVKAYNSKDAAGVASKYMDDAAIFPLDMARVDGRQ
NIQKLWQGLMDMGVSEPKLTTLDVQESGDFAFESGSISLKGPGKDSKLV
DIAGKYVEVWRKGQDGGWKLYRTIANLDPAK
HH22:
(SEQ ID NO: 27)
GQSAKEAIEAALADFVKAFNGKDAADVASKYMDDAAIFPLDMARVDGRQ
NIQKLWQGLMDTGVSEPKFTTLVVQESGDFAFESGSFSLKGPGPDSKLV
DIAGKYVEVWRKGQDGGWKLYHTIANLDPAK
HH24 (Tightest measured binder):
(SEQ ID NO: 28)
GQSAKEAIEAALADFVKAYNSKDAAGVASKYMDDAAIFPLDMAPVDGRQ
NIQKLWQGLMDMGVSEPKFTTLNVQESGDFAFESGSFSLKGPGKDSKLV
DIAGIYVEVWRKGQDGGWKLYRTIANLDPAK
HH35v1 (CDL2.1):
(SEQ ID NO: 29)
DQSAKEAIEAALADFVKVYNSKDAAGVASKYMDDAAIFPLDMAPVDGRQ
NIQKLWQGLMDMGVSEPKFTTLNVQKSGDFAFESGSFSLKGPGKDSKLV
DIAGIYVEVWRKGQDGGWKLYRTIANLDPAK
J1C-16 (CDL2.2); slightly truncated from HH35.v1/
CDL2.1
(SEQ ID NO: 30)
QSAKEAIEAALADFVKVYNSKDAAGVASKYMDDAAIFPLDMAPVDGRQN
IQKLWQGLMDMGVSEPKFTTLNVQKSGDFAFESGSFSLKGPGKDSKLVG
IAGIYVEVWRKGQDGGWKLYRTIANLGP
HH35v2:
(SEQ ID NO: 31)
DQSAKEAIEAALADFVKVYNSKDAAGVASKYMDDAVIFPLDMAPVDGRQ
NSQKLWQGLMDMGVSEPKFTTLNVQKSGDFAFESGSFSLKGPGKDSKLV
DIAGLYVEVWRKGQDGGWKLYRTIANLDPAK
W1v1:
(SEQ ID NO: 32)
GQSAKEAIEAALADFVKAYNSKDAAGVASKYMDGAAIFPLDMAPVDGRQ
NIQKLWQGLIDMGVSGVKLTTLDVQESGDFAFESGSFSLKGPDKDSKLV
DIAGKYVEVWRKGQDGGWKLYRTIANLDPAK
W1v2:
(SEQ ID NO: 33)
GQSAKEAIEAALADFLKAYNSKDAAGVASKYMDDAAIFPLDMARVDGRQ
YIQRLWQGLMDMGVSEPKFTTLNVQESGDFAFESGSFSLKGPGKDSKLV
DIAGKYVENWRKGQDGGWKLYRTIANLDPAK
W19v1:
(SEQ ID NO: 34)
LPTAHEAIEAALADFVKVYNSKDAAGVASKYMDDAVIFPLDMARVDGRQ
NIQKLWQGLMDMGVSEPKFTTLNVQESGDFAFESGSFSLKGPGKDSKLV
DIAGIYVEVWRKGQDGGWKLYRTIANLDPAR
W19v2:
(SEQ ID NO: 35)
LPTAHEAIEAALADFVKVYNSKDAAGVASKYMDDAVIFPLDMARVDGRQ
NIQKLWQGLMDMGVSEPKFTILNVQESGDFAYESGSFSLKGPGKDSKLV
DIAGIYVEVWRKGQDGGWKLYRTIANLDPAK
W24:
(SEQ ID NO: 36)
GQSAKEAIEAALADFVKAYNSKDAAGVASKYMDDAAIFPLDMAPVDGRQ
NIQKLWQGLMDMGVSEPKFTTMNVQESGDFAFESGRFSLKGPGKDSKLV
DIAGKYVEVWRKGQGGGWKLYRTIANLDPAK.

These additional sequences were obtained during the evolution of the initial design into its final form. They were sequenced from library pools that showed a significant binding signal via yeast surface display but were not characterized further:

(SEQ ID NO: 37)
GQSAKEAIEAALADFVKAYNSKDAAGVASKYMDDAAIFPLDMAPVDGRQNI
QKLWQGLMDMGVSEPKFTTLNVQESGDFAFESGSFSLKGPGKDSKLVDIAGI
YVEVWRKGQDGGWKLYRFIANLDPAK
(SEQ ID NO: 38)
GQSAKEAIEAALADFVKAYNSKDAAGVASKYMDDAAIFPLDMAPVDGRQNI
QKLWQGLIDMGVSGVKLTTLDVQESGDFAFESGSFSLKGPGKDSKLVDNAG
KYVENAVRKGQDGGWKLYRTIANLDPAK
(SEQ ID NO: 39)
DQSAKEAIEAALADFVKVYNSKDAAGVASKYMDDAVIFPLDMARVDGRQNI
QKUNQGLMDNIGVSEPKETTLNVQESGDFAFESGSFSLKCIPGKDSKINDIAGI
YVEVWRKGQDGGWRILYRTIANLDPAK
(SEQ ID NO: 40)
GQSAKEAIEAALADFVKAYNSKDAAGVASKYMDDAAIFPLDMAPVDGRQNI
QMINVQGIAIDNIGVSEPKYVITNNIQESGDFAFESGSFRLKGPGKDSKINDIAGI
YVEVWRKGQDGGWRILYRTIANLDPAK
(SEQ ID NO: 41)
GQSAKEAIEAALADFVKVYNSKDAAGVASKYMDDAAIFPLDMAPVDGRQNI
QKUNQGLMDNIGVSEPKFITIAVQESGDFAFESGSFSLKCIPGKDSKINDIAGI
YVEVWRKGQDGGWKLYRTIANLDPAK
(SEQ ID NO: 42)
DQSAKEAIEAALADFVKVYNSKDAAGVASKYMDDAVIFPLDMARVDGRQNI
QKUNQGLMDNIGVSEPKFITIAVQESGDFAFESGSFSLKCIPGKDSKINDIAGI
YVEVWRKGQDGGWKLYRTTANLDPAK
(SEQ ID NO: 43)
LPTAHEAIEAALADFVKVYNSKDAAGVASKYIVIDDAVIFPLDMARVDGRQNI
QKLWQGLMDNIGVSEPKFITIAVQESGDFAFESGSFSLKCIPGKDSKINDIAGI
YVEVWRKGQDGGWRILYRTIANLDPAK
(SEQ ID NO: 44)
GQSAKEAIEATLADEVKAYNSKDAAGVASKYMDDAAIFPLDMAPVGGRQN1
QKUNQGLMDNIGVSEPKETTLNVQESGDFAFESGSFSLKCIPGKDSKINDIAGI
YVEVWRKGQDGGWRILYRTIANLDPAK
(SEQ ID NO: 45)
GQSAKEAIEAALADFVKAYNSKDAAGVASKYMDDAAIFPLDMAPVDGRQNI
QMINVQGIAIDNIGVSGVKITTLDVQENCIDEAFESGSFSIKGPGKDSKINDIAG
KYVENAVRKGQGGGWKLYRTIANLDPVK
(SEQ ID NO: 46)
GQSAKEAIEAALADFVKAYNSKDAAGVASKYMDDAAIFPLDMAPVDGRQNI
QMINVQGLMDNIGVSEPKLTTLNVQESGDFAFESGSFSLKGPGKDSKINDIAGI
YVEVWRKGQDGGWRILYRTIANLDPAK
(SEQ ID NO: 47)
GQSAKEAIEAALADFVKAYNSKDAAGVASKYMDDAAIFPLDMAPVDGRQNI
QKLWQGLMDNIGVSEPKFITIAVQESGDFAFESGSFSLKCIPGKDSKINDIAGI
YEENIVRKGQDGCAVKLYRTIANLDPAK
(SEQ ID NO: 48)
GQSAKVAIEAALADFVKVYKSKDVAGVASKYMDDAVIFPLDNIAPVDGRQNI
QKLWQGLMDNIGVSEPKFITIAVQESGDFAFESGSFSLKCIPGKDSKINDIAGI
YVEVWRKGQDGGWRILYRTIANLDPAK
(SEQ ID NO: 49)
GQSAKEVIEAALADFVKAYNSKDAAGVASKYMDDAAIFPLDMAPVDGRQNI
QIKILAVQGLMDMGVSEPKFTILNVQESGDFAFESGSFSLKGPGKDSKINDIAGI
YVEVWRKGQDGGWKLYRTIANLDPAK
(SEQ ID NO: 50)
DQSAKEPIEAALADFVKGYNSKDAAGVASKYMDDAVIFPLDMARVDGRQNI
QIKILAVQGLMDMGVSEPKFITLNVQESGDFAFESGSFSLKGPGKDSKINDIAGI
YVEVWRKGQDGGWKLYRTIANLDPAK
(SEQ ID NO: 51)
GQSAKEAIEAALADFVKAYNSKDAAGVASKYMDDAAIFPLDMARVDGRQNI
QKLWQGLMDMGVSHKLTTLDVQESGDFAFESGSFSLKGPGKDSKLVDIAG
KYVEVWRKGQDGGWKLYRTIANLDPAK
(SEQ ID NO: 52)
GQSAKEAIEAALADFVKAYNSKDAAGVASKYMDDAAIFPLDMARVDGRQNI
QKLWQGLMDMGVSELKSTTLDVQESGDFAFESGSFSLKGPGKDSKLVDVAG
KYVVVWRKGQDGGWKLYRTISNLDPAK
(SEQ ID NO: 53)
GQSAKFAIEAALADFVKAYNSKDAAGVASKYVDDAAIFPLDMARVDGRQNI
QKLWQGLMDMGVSELKSTTLDVQESGDFAFESGSFSLKGPGKDSKLVDVAG
KYVVVWRKGQDGGWKLYRTISNLDPAK
(SEQ ID NO: 54)
GQSAKEAIEAALADFVKAYNSKDAAGVASKYMDDAAIYPLDMARVDGRQNI
QKLWQGLMDMGVSELKSTILDVQESGDFAFESGSFSIKGPGKDSKINDVAG
KYVVVWRKGQDGGWKLYRTISNLDPAK
(SEQ ID NO: 55)
GQSAKEAIEAALADFVKAYNSKDAAGVASKYMDDAAIFPLDMARVDGRQNI
QRLWQGLMDMGVSELKSTTLDVQESGDFAYESGSFSLKGPGKDSKLVDVAG
KYVVVWRKGQDGGWKLYRTISNLDPAK
(SEQ ID NO: 56)
GQSAKEAIEAALADFVKAYNSKDAAGVASKYMDDAAIFPLDMARVDGRQNI
QKLWQGLMDMGVSELKSTTLDVQESGDFAFESGSFSLKGPGKDSKLVDVAG
KYVEVWRKGQDGGWKLYRTISNLDPAK
(SEQ ID NO: 57)
GQSAKEAIEAALADFVKAYNSKDAAGVASKYMDDAAIFPLDMARVDGRQNI
QKLWQGLMDMGVSEPKLTTIDVQESGDFAFESGSISLKGPGKDSKINDIAGK
YVEVWRKGQDGGWKLYRTIANLDPAK
(SEQ ID NO: 58)
GQSAKEAIEAALADFVKAYNSKDAAGVASKYMDDAAIFPLDMARVDGRQNI
QKLWQGLMDMGVSEPKLTTIDVQESGDFAFESGSFSLKGPGKDN KINDIAG
KYVEVWRKGQDGGWKLYRTIANLDPAK
(SEQ ID NO: 59)
GQSAKEAIEAALADFVKAYNSKDAAGVASKYMDDAAIFPLDMARVDGRQNI
QKLWQGLMDMGVSEPKLTTIDVQESGDFAFESGSFSLKGPGKDSKINDIAG
KYVEVWRKGQDGGWKLYRTIANLDPAK
(SEQ ID NO: 60)
GQSAKEAIEAALADFVKAYNSKDAAGLASKYMDDAAIFPLDMAINDGRQN1
QMINVQGIAIDIMIGVSGVKITTLDVQESGDFAFESGSFSLKGPGKDSKINDIAG
KYVENWRKGQDGGWKLYRTIANLDPAK
(SEQ ID NO: 61)
GQSAKEAIEAALADFVKAYNSKDAAGVASKYMDDAMFPLDMARVDGRQNI
QMINVQGLMDMGVSEPKLTALDNIQESGDFAFESGSFSLKGPGKDSKINDIAG
KYVENWRKGQDGGWKLYRTIANLDPAK
(SEQ ID NO: 62)
GQSAKEAIEAALADFVKSYNSKDAAGVASKYMDDAMFPLDMAPVDGRQNI
QMINVQGLMDMGVSGLKLTTLDVQESGDFAFESGSFSLKGPGRDSKININFG
KYVENAVRKGQDGGWKLYRTIANLDPAK
(SEQ ID NO: 63)
GQSAKEAIEAALADFVKAYNSKDAAGVASKYMDDAAIFPLDMAPVDGRQNI
QKLWQGIAIDINGVSGVKITTLDVQESGDFAFESGSFSLKGPGKDSKINDIAG
KYVENWRKGQDGGWKLYRTIANLDPAK
(SEQ ID NO: 64)
GQIAKEAIEAALADFVKAYNSKDAAGVVSKYAIDDAAIFPLDMAPVDGRQNI
QKLWQGIAIDINGVSGVKITTLDVQESGDFAFESGSFSLKGPGKDSKINDIAG
KYVENWRKGQDGDWKLYRTIANLDPAK
(SEQ ID NO: 65)
GQSAKEMEAALADFVKAYNSKDAAGVASKYTDDAAIFPLDMAPVDGRQM
QKLWQGIAIDINGVSGVKITTLDVQESGDFAFESGSFSLKGPGKDSKINDIAG
KYVENWRKGQDGGWKLYRTIANLDPAK
(SEQ ID NO: 66)
GQSAKEAIEAALADFVKVYNSKDAAGVAGKYMDDAAIFPLDMAPVDGRQNI
QKLWQGIAIDINGVSGVKITTLDVQESGDFAFESGSFSLKGPGKDSKINDIAG
KYVENWRKGQDGGWKLYRTIANLDPAK
(SEQ ID NO: 67)
GQSAKEAIEAALADFVKAYNSKDAAGVASKYMDDAMFPLDMARVDGRQDI
QMINVQGLMDMGVSEPKLTTLDVQESGDFAFESGSFSLKGPGKDSKINDIAG
KYVENAVRKGQDGGWKLYRTIANLDPAK
(SEQ ID NO: 68)
GQSAKEAIEAALADFVKAYNSKDAAGVASKYMDDAAIFPLDMARVDGRQNI
QMINVQGLMDMGVSEPKLTTLDVQESGDFAFESGSFSLKGPGKDSKINDIAG
KYVENAVRKGKDGGWKLYRTIANLDPAK
(SEQ ID NO: 69)
GQSAKEAIEAALADFVKAYNSKDAAGVASKYMDDAAIFPLDMARVDGRQNI
QKLWQGLMDMGVSEPKLTTLDVQESGDFAFESGSFSLKGPGKDSKINDIAG
KYVENAVRKGQNGGWKLYRTIANLDPAK
(SEQ ID NO: 70)
GQNAKEAIEAALADFVKAYNSKDAAGVASKYMDDAAIFPLDMAPVDGRQNI
QKLWQGIAIDINGVSGVKITTLDVQESGDFAFESGSFSLKGPGKDSKINDIAG
KYVENWRKGQDGGWKLYRTIANLDPAK
(SEQ ID NO: 71)
AQSAKEAIEAALADFVKAYNSKDAAGVASKYMDDAAIFPLDMAPVDGRQNI
QKLWQGLNIDMGVSGVKLTTLDVQESGDFAFESGSFSIKGPGKDSKINDIAG
KYVEVWRKGQDGGRKLYRTIANLDPAK
(SEQ ID NO: 72)
GQSAKEAIEAALADFVKAYNSKDAAGVASKYMDDAAIFPLDMAPVDGHQNI
QMINVQGLNIDNIGVSGVKLTTLDVQESGDFAFESGSFSIKGPGKDSKINDIAG
KYVENWRKGQDGDWKLYRTIANIARR
(SEQ ID NO: 73)
GQSAKEAIEAALADFVKAYNSKDAAGVASKYMDDAAIFPLDMARVDGRQNI
WINVQGLMDMGVSEPKLTTLDVQESGDFAFESGSFSLKGPGKDSKINDNAG
KYVEVWRKGQDGGWKLYRTIANLDPAK
(SEQ ID NO: 74)
GQSAKEAIEAALADFVKAYNSKDAAGVARKYMDDAAIFPLDMARVDGRQNI
QKLWQGLMDMGVSEPKLTTLDVQESGDFAFESGSFSLKGPGKDSKINDNAG
KYVEVWRKGQDGGWKLYRTIANLDPAK
(SEQ ID NO: 75)
GQSAKEAIEAALADFVKAYNSKDAAGVASKYMDDAAIFPLDMAPVDGRQNI
QKLWQGLNIDMGVSGVKLTTLDVQESGDFAFESGNFSLKGPGKDSKLVDIAG
KYVEVWRKGQDGGWKLYRTIANLDPAK
(SEQ ID NO: 76)
GQSAKEAIEAALADFVKAYNSKDAAGVASKYMDDAAIFPLDMARVDGRQNI
QMINVQGIADMGVSEPKITTLDVQESGDFAFESGSFSIKGPGKDSKINDIAGK
YVEVWRKGQDGGWRILYRTIANLDPAK
(SEQ ID NO: 77)
GQSAKEAIEAALADFVKAYNSKDAAGVASKYMDDAAIFPLDMARVDGRQNI
QMINVQGLNIDNIGVSEPKLTTLDVQESGDFVFESGSFSLKGPGKDSKINDIAG
KYVEVWRKGQDGGWKLYRTIANLDPAK
(SEQ ID NO: 78)
GQIAKEAIEAALADFVKAYNSKDAAGVASKYMDDAAIFPLDNIARVDGRQNI
QKLWQGLMDMGVSEPKLTTLDVQESGDFAFESGSFSLKGPGKDSKLVDIAG
KYVEVWRKGQDGGWKLYRTIANLDPAK
(SEQ ID NO: 79)
GQSAKEAIEAALADFVKAYNSKDAAGVASKYMDDAAIFPLDMARVDGRQNI
QMINVQGLNIDTGVSEPKITTLDVQESGDFAFESGSFSIKGPGKDSKINDIAGK
YVEVWRKGQDGGWRILYRTIANLDPAK
(SEQ ID NO: 80)
GQSAKEAIEAALADFVKAYNSKDAAGVASKYMDDAAIFPLDMARVDGRQNI
QMINVQGLMDMCVSEPIKUVILDVQESGVEAFESGSFSLKGPGKDSKINDIAG
KYVEVWRKGQDGGWKLYRTIANLDPAK
(SEQ ID NO: 81)
GQSAKEAIEAALADFVKAYNSKDAAGVASKYMDDAAIFPLDMAPVDGRQNI
QKLWQGIAIDMGVSGVKITTLDVQESGDFAFESGSFSIKGPGKDSKINDIAG
KYVGIAVRKGQDGGWKLYRTIANLDPAK
(SEQ ID NO: 82)
GQSAKEAIEAALADFVKAYNSKDAAGVASKYMDDAAIFPLDMAPVDGRQNI
WINVQGLNIDMGVSGVKLIILDVQESGDFTFESGSFSLKGPGKDSKINDIAG
KYVENWRKGQDGGWKLYRTIANLDPAK
(SEQ ID NO: 83)
GQSAKEAIEAALADFVKAYNSKDAAGVASKYMDDAAIFPLDMARVDGRQNI
QMINVQGLMDMGVSEPKLTTIDVQESGDFAFESGSFRIKGPGKDSKINDIAG
KYVENWRKGQDGGWKLYRTIANLDPAK
(SEQ ID NO: 84)
GQSAKEAIESALADFVKVYNSKDAAGVASKYMDDAAIFPLDMAPVDGRQNI
QMINVQGIAMMGVSGVKITTLDVQESGDFAFESGSFSIKGPGKDSKINDIAG
KYVENWRKGQDGGWKLYRTIANLDPAK
(SEQ ID NO: 85)
GQSAKEAIEAALADFVKAYNSKDAAGVASKYMDDAAIFPLDMARVDGRQNI
QKLWQGLMDMGVSEPKLTTLDVQESGDFAFESGSFSLKGPGKDSKLNDIAG
KYVEVWRKGQDGGWKLYRTIANLDPAK
(SEQ ID NO: 86)
GQSAKEAIEAALADFVKAYNSKDAAGVASKYMDDAAIFPLDMAPVDGRQNI
QKLWQGLNIDLGVSEPKFTTLDVQESGDFAFESGSFSLKGPGQDSKLVDIAGK
FVEVWRKGQDGGWKLYRTIANLDPAK
(SEQ ID NO: 87)
GQSAKETIEAALADFATKAYNSKDAAGVASKYMDDAAIFPLDMAPVDGRQN1
QMINVQGIAMMGVSGVKITTLDVQESGDFAFESGSFSIKGPGKDSKINDIAG
KYVENWRKGQDGGWKLYRTIANLDPAK
(SEQ ID NO: 88)
GQSAKEAIEAALADFVKAYNSKDAAGVASKYMDDAAIFPLGMARVDGRQNI
QKLWQGLMDMGVSELKSTTLDVQESGDFAFESGSFSLKGPGKDSKIJVDVAG
KYVVIAVRKGQDGGWKLYRTISNLDPAK
(SEQ ID NO: 89)
GQSAKEAIEAALADLVKAYNSKDAAGVASKYMDDAAIFPLDMARVDGRQNI
QKLWQGLMDMGVSELKSTTIDVQESGDFAFESGSFSLKGPGKDSKINDVAG
KYVVIAVRKGQDGGWKLYRTISNLDPAK
(SEQ ID NO: 90)
GQSAKEAIEAALADFVKAYNSKDAAGVASKYMDDAAIFPLDMARVDGRQNI
QKLWQGLMDMGVSELKSTTIDVQESGDFAFESGSFSLKGPGKDSKINDVAG
KYVFNWRKGQDGGWKLYRTINLDPAK
(SEQ ID NO: 91)
GQSAKEAIEAALADFVKAYNSKDAAGVASKYMDDAAIFPLDMVRVDGRQNI
QKLWQGLMDMGVSELKSTTIDVQESGDFAFESGSFSLKGPGKDSKINDVAG
KYVENWRKGQDGGWKLYRTISNLDPAK
(SEQ ID NO: 92)
GQSAKEAIEAALADFVKAYNSKDAAGVASKYLDDAAIFPLDMARVDGRQNI
QMINVQGLMDMGVSGPIKFTILDVQESCIDFAFESGSFSIKGPCIKDSKINDVAG
KYVVIAVRKGQDGGWKLYRTISNLDPAK
(SEQ ID NO: 93)
GQSAKEAIEAALADFVKAYNSKDAAGVASKYMDDAAIFPLDMARVDGRQDI
QKLWQGLMDMGVSELKSTTLDVQESGDFAFESGSFSLKGPGKDSKIJVDVAG
KYVVIAVRKGQDGGWKLYRTISNLDPAK
(SEQ ID NO: 94)
GQSAKEAIEAALADFVKAYNSKDAAGVASKYMDDAAIFPLDMARVDGRQNI
QKLWQGLMDMGVSELKLTTLDVQESGDFAFESGSFSLKGPGKDSKLVDVAG
KYVVIAVRKGQDGGWKLYRTISNLDPAK
(SEQ ID NO: 95)
GQSAKEAIEAALADFVKAYNSKDAAGVASKYMDDAAIFPLDMARVDGRQNI
QKLWQGLMDMGVSELKSTTLDVQESGDFAFESGSFSLKGPGKDSKMVDVA
GKYVVVIAIRKGQDGGWKIARFISNLDPAK
(SEQ ID NO: 96)
GQSAKEAIEAALADFVKAYNSKDAAGVASKYMDDAAIFPLDMARVDGRQNI
QKLWQGLMDMGVSELKSTTLDVQESGDFAFESGSFSLKGPGKDSKINDVVG
KYVVIAVRKGQDGGWKLYRTISNLDPAK
(SEQ ID NO: 97)
GQSAKEAIEAALADFVKAYNSKDAAGVASKYMDDAAIFPLDMARVDGRQNI
QKUNQGLMDMGVSELKSTTLDVQESGDFAFESGSFSLKGPGKDSKIADVAG
KYVVIAVRKGQDGGWKLYRTISNLDPAK
(SEQ ID NO: 98)
GQSAKEAIEAALADFVKAYNSKDAAGVASKYMDDAAIFPLDMARVDGRQNI
QKIAVQGLNIDMGVSEPKLTTLDVQESGDFAFESGSFSLKGPGKDSKINDIAG
KYVFNWRKGQDGGWKLYRTIANLGPAK
(SEQ ID NO: 99)
GQSAKEAIEAALADFVKAYNSKDAAGVASKYMDDAAIFPLDMARVDGRQNI
QKLWQGLNIDMGVSELKSTTLDVQESGDFAFESGSFSLKGPGKDGEINDVAG
KYVVIAVRKGQDGGWKLYRTISNLDPAK
(SEQ ID NO: 100)
GQSAKEAIEAALADFVKAYNSKDAAGVASKYMDDAAIFPLDMARVDGRQNI
QKLWQGLMDMGMSEIKSTTLDVQESGDFAFESGSFSLKGPGKDSKIVDVAG
KYVVIAVRKGQDGGWKLYRTISNLDPAK
(SEQ ID NO: 101)
GQSAKEAIEAALADFVKAYNSKDAAGVASKYMDDAAIFPLDMARVDGRQNI
QKLWQGLMDMGVSEPKSTTLDVQESGDFAFESGSFSLKGPGKDSKINDIAG
KYVFNWRKGQDGGWKLYRTIANLDPAK
(SEQ ID NO: 102)
GQSAKEAIEAALADFVKAYNSKDAAGVASKYMDDAAIFPLDMARVDGRQNI
QKLWQGLMDMGVSEPKFTTINVQESGDFAFESGSFSLKGPGKDSKLVDIAG
KYVVIAVRKGQDGGWKLYRTISNLDPAK
(SEQ ID NO: 103)
GQSAKEAIEAALADFVKAYNSKDAAGVASKYMDDAAIFPLDMARVDGRQNI
IKLWGIAMMGVSELKSTILDVQESGDFAFESGSFSIKGPGKDSKINDVAG
KYVVIAVRKGQDGGWKLYRTISNLDPAK
(SEQ ID NO: 104)
GQSAKEAIEAALADFVKAYNGKDAAGVASKYNIDDAAIFPLDMARVDGRQNI
QKIINVQGLMDMGVSELKSTTLDVQESGDFAFESGSFSLKGPGKDNKLVDAG
KYVEVWRKGQDGGWKLYRTISNLDPAK
(SEQ ID NO: 105)
GQSAKEAIEAALADFVKAYNSKDAAGVASKYIDDAAIFPLDMARVDGRQNIQ
KLWQGLMDMGVSEPKLTTLDVQESGDFAFESGSFSLKGPGKDSKLVDIAGK
YVEVWRKGQDGGWKLYRTIANLDPAK
(SEQ ID NO: 106)
GQSAKEAIEAALADFVKAYNSKDAAGVASKYNIDDAAIFPLDMARVDGRQNI
QKLWQGLMDMGVSEPKLTTLDVQESGDFAFESGSFSLKGPGKDSKLVDIAG
KYVEVWRKGQDGGWKLYRTIANLDPSK
(SEQ ID NO: 107)
GQSAKEAIEAALADFVKAYNSKDAADVASKYMDDAAIFPLDMAPVDGRQNI
QKLWQGLMDMGVSEPKLTTLDVQESGDFAFESGSFSLKGPGKDSKLVDIAG
KYVEVWRKGQDGGWKLYRTIANLDPAK
(SEQ ID NO: 108)
GQRAKEAIEAALADFVKAYNSKDAAGVASKYMDDAAIFPLDNIAPVDGRQNI
QKIINVQGIAIDMGVSGVKITTLDVQESGDFAFESGSFSIKGPGKDSKINDIAG
KYVEVWRKGQDGGWKLYRTIANLDPAK
(SEQ ID NO: 109)
GQSAKEMEAALADFVKAYNSKDAAGVASKYNIDDAAIFPLDMASVDGRQNI
QKILAVQGIAIDMOVSGVKITTLDVQESGDFAFESGSFSIKGPGKDSKINDIAG
KYVEVWRKGQDGGWKLYRTIANLDPAK
(SEQ ID NO: 110)
GQSSKEAIEAALADFVKAYNSKDAAGVANKYNIDDAAIFPLDMARVDGRQNI
QKIINVQGLNIDMGVSEPKLTTLDVQESGDFAFESGSFSLKGPGKDSKINDIAG
KYVEVWRKGQDGGWKLYRTIANLDPTK
(SEQ ID NO: 111)
GQSAKEAIEAALADFVKAYNSKDAAGVASKYMDDAAIFPLDMAPVDGRQNI
QKILAVQGIAIDMOVSGVKITTLDVQESGDFAFESGSFSIKGPGKDSKINDIAG
KYVEVWRKGQDGGWKLYRTIANLPG
(SEQ ID NO: 112)
GQSAKEAIEAALADFVKAYNSKDAAGVASKYMDDAAIFPLDMAPVDGRQNI
QKIINVQGLNIDMGVSGVKLTTLDVQESGDFAFESGSFSIKGPGDSKINDIAGK
YVEVWRKGQDGGWRILYRTIANLDPAK
(SEQ ID NO: 113)
GQSAKEAIEAALAEFVKAYNCKDAAGVASKYNIDDAAIFPLDMARVDGRQN1
QKLWQGLMDMGVSEPELTTLDVQESGDFAFESGSFSLKGPGKDSKLVDIAGK
YVEVWRKGQDGGWRILYRTIANLDPAK
(SEQ ID NO: 114)
SQSAKETIEAALADFVKAYNSKDAAGVASKYMDDAEIFPLDMARVDGRQNI
QKILAVQGIAIDMGVSEPKLTTLDVQESGDFAFESGSFSLKGPGKDSKINDIAG
KYVEVWRKGQDGGWKLYRTIANLDPAK
(SEQ ID NO: 115)
GQSAKEAIEAALADFVKAYNSKDAAGVASKYMDDAAIFPLDMARVDGRQNI
QKIAVQGLMDMGVSELKUITILDVQESCIDEAFESGSFSIKGPCIKDSKINDVAG
KYVMVAVRKGQDGGWKLYRTISNLDPAK
(SEQ ID NO: 116)
GQSAKEAIEAALADFVKAYNSKDAAGVASKYMDDAAIFPLDMARVDGRQNI
QKIAVQGLMDMGVSEPKLTTLGVQESGDENFESCISFSLKGPGKDSKINDIAG
KYVFNWRKGQDGGWKLYRTIANLDPAK
(SEQ ID NO: 117)
GQSAKEAIEAALADFVKAYNSKDAAGVASKYMDDAAIFPLDMARVDGRQNI
QKLWQGLMDMGVSEPKLTTLDVQESGDFAFESGSFSLKGPGKDSKLVDIAG
KYVEVWRKGQDGGWKLYRTISNLDPAK
(SEQ ID NO: 118)
GQSAKEAIEAALADFVKAYNSKDAAGVASKYMDDAAIFPLDMARVDGRQNI
QKLWQGLMDMGVSEPKLTTLDVQESGDFAFESGNFSLKGPGKDSKINDIAG
KYVFNWRKGQDGGWKLYRTIANLDPAK
(SEQ ID NO: 119)
GQSAKEAIEAALADFVKAYNSKDAAGVASKYMDDAAIFPLDMARVDGRQNI
QKIAVQGLTDMGVSELKUTILDVQESGDFAFESGSFSLKCIPGKDSKINDVAG
KYVENWRKGQDGGWKLYRTISNLDPAK
(SEQ ID NO: 120)
GQSAKEAIEAALADFVKAYNSKDAAGVASKYMDDAAIFPLDMARVDGRQNI
QKIAVQGLMDMGVSEPKLTTLDVQESGYFAFESCISFSLKGPGKDSKINDIAG
KYVFNWRKGQDGGWKLYRTIANLDPAK
(SEQ ID NO: 121)
GQSAEEAIEAALAEFVKAYNSKDAAGVASKYMDDAAIFPLDMARVDGRQNI
QKIAVQGLMDMGVSEPKLTTLDVQESGDENFESCISFSLKGPGKDSKINDIAG
KYVFNWRKGQDGGWKLYRTIANLDPAK
(SEQ ID NO: 122)
GQSAKEAIEAALADFVKAYNSKDAAGVVSKYMDDAAIFPLDMARVDGRQNI
QKLWQGLMDMGVSEPKLTTLDVQESGDFAFESGSFSLKGPGKDSKLVDIAG
KYVEVWRKGQDGGWKLYRTIANLDPAK
(SEQ ID NO: 123)
GQSAKFAIKAALADINKAYNSKDAAGVASKYNIDDAAIFPLDMARVDGRQNI
QKLWQGLMDMGVSELKLITLDVQESCIDEAFESGSFSIKGPCIKDSKINDVAG
KYVENWRKGQDGGWKLYRTISNLDPAK
(SEQ ID NO: 124)
GQSAKEAIEAALADFVKAYNSKDAAGVASKYMDDAAIFPLDMARVVGRQNI
QKLWQGLMDMGVSEPKFTTLDVQESGDFAFESGSFSLKGPGQDSKLVDIAG
KYVFNWRKGQDGGWKLYRTIANLDPAK
(SEQ ID NO: 125)
GQSAKEAIEAALADFVKGYNPKDGAGVASKSMDDAPIFPPDMARVDGRQNI
QKLWQGLNIDTGVSEPKFTTLDVQESGDFAFESGSFSLKGPGPDSKINDIAGK
YVVVWRKGQDGGWKLYRTISNLDPAK
(SEQ ID NO: 126)
GQSAKEAIEAALADFVKAYNSKDAAGVASKYMDGAAIFPLDMARVDGRQNI
QKUNQGLMDNIGVSELKUITILDVQESCIDFAFESGSFSIKGPCIKDSKINDVAG
KYVVIAVRKGQDGGWKLYRTISNLDPAK
(SEQ ID NO: 127)
GQSAKEAIEAALADFVKAYNSKDAAGVASKYMDDAAIFPLDMARVDGRQNI
QKUNQGLMDNIGVSELKUITILDVQESCIDFAFESGSFSIKGPCIKDSKINGVAG
KYVENWRKGQDGGWKLYRTISNLDPAK
(SEQ ID NO: 128)
GQSAKEAIEAALADFLKGYNPKDGAGVASKYMDDAPIFPPDMAPVDGPQNIL
KLWQGLMDMGVSGPKFTTLVVQESGDFAFESGSFSPKGPGKDSKLVDIAGK
YVVVWRKGQDGGWKLYRTISNLDPAK
(SEQ ID NO: 129)
GQSAKEAIEAALADFAKVYNGKDGAGVASKSMDDAPIFPPDMATIVDGPQNI
LKLWQGLMDMGVSEPKFTTLVVQESGDFAFESGSFSVKGPGTDSKINDIAGK
YVVVWRKGQDGGWKLYRTISNLDPAK
(SEQ ID NO: 130)
GQSAKEAIEAALADFVKGYNRKDGAGVASKSMDDAPIFPLDMATIVDGPQNI
IKLWQGIAIDIGNISEPKFTTINVQESGDFAFESGSFSVKGPGPDSKINDIAGK
YVVVWRKGQDGGWKLYRTISNLDPAK
(SEQ ID NO: 131)
GQSAKEAIEAALADFVKAYNSKDAAGVASKYMDDAAIFPLDMARVDGRQNI
QKIAVQGLMDNIGVSELKLITMDVQESGDFAFESCISFSLKGPGKDSKINDVA
GKYVVVWRKGQDGGWRILYRTISNLDPAK
(SEQ ID NO: 132)
GQSAKEAIEAALADFVKAYNSKDAAGVASKYMDDAAIFPLDMALVDGRQNI
QKIAVQGLNIDNIGVSGVKITTLDVQESGDFAFEGGSFSIKGPGKDSKINDIAG
KYVFNWRKGQDGGWKLYRTIANLDPAK
(SEQ ID NO: 133)
GQSAKEAIEAALADFVKAYNSNDATGVASKYMDDAAIFPLDMAPVDGRQNI
QKLWQGLNIDMGVSGVKLTTLDVQESGDFAFESGSFSLKGPGKDSKINDIAG
KYVFNWRKGQDGGWKLYRTIANLDPAK
(SEQ ID NO: 134)
GQSAKEAIEAALADFVKAYNSKDAAGVASKYMDDAAIFPLDMAPVDGRQNI
QKLWQGLNIDMGVSGVKLTTLDVQESGDFAFESGSFSLKGPGNDSKINDIAG
KFVEVIVRKGQDGGWKLYRTIANLDPAK
(SEQ ID NO: 135)
GQSAKEAIEAALADFVKAYNSKDAAGVASKYMDDAAIFPLDMAPVDGRQNI
QKIAVQGLKDMGVSGVKLTILDNIQESGDFAFESGSFSLKCIPGKDSKINMAG
KYVFNWRKGQDGGWKLYRTIANLDPAK
(SEQ ID NO: 136)
GQSAKEAIEAALADFVKAYNSKDAAGVASKYMDDAAIFPLDMARVDGRQNI
QKLWQMNIDNIGVSEPKLTTIDVQESGDFAFESCISFSLKGPGKDSKINDIAG
KYVFNWRKGQDGGWKLYRTIANLDPAK
(SEQ ID NO: 137)
GQSAKEAIEAALADFVKAYNSKDAAGVASKYMDDAAIFPLDMAPVDGCQNI
QKLWQGLMDMGVSGVKLTTLDVQESGDFAFESGSFSIKGPGKDSKLVDIAG
KYVEVWRKGQDGGWKLYRTIANLDPAK
(SEQ ID NO: 138)
GQSAKEAIEAALADFVKAYNSKDAAGVASKYTDDAAIFPLDMARVDGRQNI
QKLWQGLMDMGVSEPKLTPLDVQESGDFAFESGSFSLKGPGKDSKLVDIAG
KYVEVWRKGQDGGWKLYRTIANLDPAK
(SEQ ID NO: 139)
GQSAKEAIEAALADFVKACNSKDAAGVASKYMDDAAIFPLDMARVDGRQNI
EKLWQGLMDMGVSEPKLTTLDVQESGDFAFESGSFSIKGPGKDSKLVDIAGK
YVEVWRKGQDGGWKLYRTIANLDPAK
(SEQ ID NO: 140)
GQSAKEAIEAALADFVKAYNSKDAAGVASKYMDDAAIFPLDMAPVDGRQNI
QKLWQGLMDMGVSGVKLTTLDVQESGDFAFESGSFSIKSPGKDSKLVDIAG
KYVEVWRKGQDGGWKLYRTIANLDPAK
(SEQ ID NO: 141)
GQSVKEAIEAALADFVKAYNSKDAAGVASKYMDDAAIFPLDMAPVDGRQNI
QKLWQGLMDMGVSGVKITTLDVQESGDFAFESGSFSIKGPGKDSKLVDIAG
KYVEVWRKGQDGGWKLYRTIANLDPAK
(SEQ ID NO: 142)
GQSAKEAlEAALADFVKAYNSKDAAGVASKYMDDAAIFPLDMARVDGRQNI
QKLWQGLMDMGVSEPKLTTLDVQESGDVAFESGSFSLKGPGKDSKLVDIAG
KYVEVWRKGQDGGWKLYRTIANLDPAK
(SEQ ID NO: 143)
GQSAKFAIEAALADFVKAYNSKDAAGVASKYKDDAAIFPLDMARVDGRQNI
QKLWQGLMDMGVSEPKLTTQDVQESGDFAFESGSFSLKGPGKDSKINDIAG
KYVEVWRNGQDGGWKLYRTIANLDPAK
(SEQ ID NO: 144)
GQSAKEAIEAALADFVKAYNSKDAAGVASKYMDDAAIFPLDMARVDGRQNI
QKLWQGLMDMGVSEPKLTTLDVQESGDFAFESGSFSLKGPGKDSKLVDIAG
KYVEVWRKGRDGGWKLYRTIANLDPAK
(SEQ ID NO: 145)
GQSAKEAIEAALADFVKAYNSKDAAGVASKYMDDAAIFPLDMARVDGRQNI
QKLWQGLMDMGVSEPKLTTLDVQESGDFAFESGSFSLKGSGKDSKLVDIAG
KYVEVWRKGQDGDWKLYRTIANLDPAK
(SEQ ID NO: 146)
GQSAKEAIEAALADFVKAYNSKDAAGVASKYMDDAAIFPLDMARVDGRQNI
QKLWQGLMDMGVSEPKLTTLDVQESGDFAFESGSFSLKGPGPDSKLVDIAGK
YVEVWRKGPDGGWKLYRTIANLDPAK
(SEQ ID NO: 147)
GQSAKEAIEAALADFVKAYNSKDAAGVASKYMDDAAIFPLDMTPVDGRQNI
QKLWQGLMDMGVSGVKITTLDVQESGDFAFESGSFSIKGPGKDSKLVDIAG
KYVEVWRKGQDGGWKLYRTIANLDPAK
(SEQ ID NO: 148)
GQSAKEAIEAALADFVKAYNSKDAAGVASKYMDDAAIFPLDMAPVDGRQNI
QKLWQGLNIDMGVSGVKLTTLDVQESGDFAFESGSFSLKGPGKDSKINDIAG
KYVFNWRKGQDGGWKLYRTIANPDPAK
(SEQ ID NO: 149)
GQSAKEAIEAALADFVKAYNSKDAAGVASKYKDDAAIFPLDMAPVDGRQNI
QKLWQGLNIDMGVSGVKSTTLDVQESGDFAFESGSTSLKGPGKDSKLVDIAG
KYVFNWRKGQDGGWKLYRTIANLDPAK
(SEQ ID NO: 150)
GQSAKEAIEAALADFVKAYNSKDAAGVASKYMDDAAIFPLGMARVDGRQNI
QKLWQGLMDMGVSEPKLTTLDVQESGDFAFESGSFSLKGPGKDSKLVDIAG
KYVEVWRKGQDGGWKLYRSIANLDPAK
(SEQ ID NO: 151)
GQSAKEAIEAALADFVKAYNSKDAAGVASKYMDDAAIFPLDMAPVDGRQNI
QKIAVQGLNIDNIGVSGVKLTTLDVQESGDFAFESGSFSIKGPGKDSKINDIAG
KYVFNWRKGQDGGWKLYRTIANPDPAE
(SEQ ID NO: 152)
GQSAKEAIEAALADFVKAYNSKDAAGVASKYMDDAAIFPLDMAPVDGRQNI
QKLWQGLNIDMGMSGVKLTTLDVQESGDFAFESGSFSLKGPGKDSKLVDIAC
KYVFNWRKGQDGGWKLYRTIANLDPAK
(SEQ ID NO: 153)
GQSAKEAIEAVLADFVKAYNSIVIDAAGVASKYMDDAAIFPLDMAPVDGRQNI
QKLWQGIAIDMGVSGVKITTLDVQESGDFAFESGSFSIKGPGKDSKINDIAG
KYVFNWRKGQDGGWKLYRTITNLDPAK
(SEQ ID NO: 154)
GQSAKEAIEAALADFVKAYNSKDAAGVASKYMDDAAIFLLDMAPVDGRQNI
QKLWQGIAIDMGVSGVKITTLDVQESGDFAFESGSFSIKGPGKDSKINDIAG
KYVFNWRKGQDGGWKLYRTIANLDPAK
(SEQ ID NO: 155)
GQSAKEAIEAALADFVKAYNSKDAAGVASKYMDDAAIFPLDMAPVDGRQNI
QKLWQGLNIDMGVSGVKLTTLDVQESGDFAFESGSFSLKGPVKDSKINDIAG
KYVFNWGKGQDGCIWKLYRTIANQDPAK
(SEQ ID NO: 156)
GQSAKEAIEAALADFVKAYNSNDAAGVASKYMDDPAIFPLDMAPVDGRQNI
QKLWQGIAIDMGVSGVKITTLDVQESGDFAFESGSFSIKGPGKDSKINDIAG
KYVFNWRKGQDGGWKLYRTIANLDPAK
(SEQ ID NO: 157)
GQSAKEAVEAALADFVICVYNSKDAAGVASKYNIDDANIFPLDMAPVDGRQN
IQKLWQGLMDMGVSGVKLTTLDVQESGDFAFESGSFSLKGPGKDSKLVDIAG
KYVFNWRKGQDGGWKLYRTIANLDPAK
(SEQ ID NO: 158)
GQSAKEAIEAALADFVKAYNSKDAAGVASKYMDDAAIFPLDMARVDGRQNI
QKLWQGLMDMGVSELKLTSLDVQESGDFAFESGSFSLKGPGKDSKLVDIAG
KYVEVWRKGQDGGWKLYRIIANLDPAK
(SEQ ID NO: 159)
GQSAKEAIEAALADFVKAYNSKDTTGVASKYAIDDAAIFPLDMAPVDGRQNI
QKLWQGLMDMGVSGVKLTTLDVQESGDFAFESGSFSLKGPGKDSKINDIAG
KYVFNWRKGQDGGWKLYRTIANLDPAK
(SEQ ID NO: 160)
GQSAKEAIEAALADFVKAYNSKDAAGVASKYMDDASIFPLDMARVDGRQNI
QKIAVQGLNIDNIGVSEPKLTTIDVQESGDENFESGSFSLKGPGKDSKINDIAG
KYVFNWRKGQDGGWKLYRTIANLDPAK
(SEQ ID NO: 161)
GQSAKEAIEAALADFVKAYNSNDAAGVASKYMDDAAIFPLDMAPVDGRQNI
QKIAVQGIAIDNIGVSGVKITELDVQESGDFAFESGSFSLKGPGKDSKINDIAG
KYVFNWRKGQDGGWKLYRTIANLDPAK
(SEQ ID NO: 162)
GQSAKEAIEAALADFVKAYNSKDAAGVASKYMDDAAIFPLDMAPVDGRQNI
QKLWQGLMDMGVSGVKLTTLDVQESGDFAFESGSFSLKGPDKDSKINDIAG
KYVFNWRKGQDGGWKLYRTIANLDPAK
(SEQ ID NO: 163)
GQSAKEAIEAALADFVKAYNSKDAAGVASKYMDDAAIFPLDMAPVDGRQNI
QKIAVQGLNIDMGVSGVKITTLDVQESGDNIAFESGSFSIKGPGKDSKINDIAG
KYVFNWRKGQDGGWKLYRTIANLDPAK
(SEQ ID NO: 164)
GQSAKEAIEAALADFVKAYNSKDAAGLASKYMDDAAIFPLDMARVDGRQNI
QKLWQGLMDMGVSEPKLTTLDVQESGDENFESGSFSLKGPGKDSKINDIAGI
YVEVWRKGQDGGWKLYRTIANLDPAK
(SEQ ID NO: 165)
GQSAKEAIEAALADFVKAYNSKDAAGVASKYMDDAAIFPLDMARVDGRQNI
QKILAVQGLNIDIVIGVSEPKLTTIDVQESGDENFESGSFSLKGPGKDSKINDIAG
KYVFNWRKGQDGGWTLYRIIANLDPAK
(SEQ ID NO: 166)
GQSAKEAIEAALADFVKAYNSKDAAGVASKYMDDAAIFPLDMAPVDGRQNI
PKAINVQGLMDMGVSGVKLTUDNIQESGDFAFESGSFSLKGPGKDSKINDIAG
KYVFNWRKGQDGGWKLYRTIANLDPAK
(SEQ ID NO: 167)
GQSAKEAIEAALADFVKAYNSKDAAGVASKYMDDAAIFPLDMAPVDGRQNI
QKIAVQGIAIDIVIGVSGVKITELDVQESGDFAFESGSFSLKGPGKDCKINDIAG
KYVKIAVRKGQDGGWKLYRTIANLDPAK
(SEQ ID NO: 168)
GQSAKEAIEAALADSVKAYNSKDAAGVASKYMDDAAIFPLDMARVDGRQNI
QKLWQGLMDMGVSEPKLTTLDVQECGDFAFESGSFSLKGPGKDSKLVD1AG
KYVFNWRKGQDGGWKLYRTIANLDPAK
(SEQ ID NO: 169)
GQSAKEAIEAALADFVKAYNSKDAAGVASKYMDDAAIFPLDMAPVDGRQNI
QKILLQGLMDMGVSGVKLITLDNIQESGDFAFESGSFSLKGPGKDSKINDIAG
KYVFNWRKGQYGGWKLYRTIANLDPAK
(SEQ ID NO: 170)
GQSAKEAIEAALADFVKAYNSKDAAGVASNNTMDDAAIFPLDMAPVDGRQNI
QKILWQGLNIDNIGVSGVKIXTLDVQESGDFAFESGSLSLKGPGKDSKINDIAG
KYVEVWRKGQDGGWKLYRTTANLDPAK
(SEQ ID NO: 171)
GQSAKEAIEAALADYVKAYNNKDAAGVASKYMDDAAIFPQDMAPVDGRQN
IQKLWQGLMDMGVSGVKLTTLDVQESGDFAFESGSFSLKGPGKDSKLVDIAG
KYVEVWRKGQDGGWKLYRTIANLDPAK
(SEQ ID NO: 172)
GQSAKEAIEAALADFVKAYNSKDAAGVASKYMDDAAIFPLDMAPVDGRQNI
QKILWQGLNIDNIGVNGNIKITTLDVQESGDFITVSGSFSIKGPGKDSKINDIAG
KYVEVWRKGQDGGWKLYRTIANLDPAK
(SEQ ID NO: 173)
GQSAKFAIEAALADEVKAYNSKDGAGVASKYNIDDAPIEPLDMARVDGRQN1
QKLWQGLNIDTGVSEPKFTTLVVQESGDFAFESGSFSPKGPGTDSKILNDIAGK
YVEVWRKGQDGGWKLYRTIANLEPAK
(SEQ ID NO: 174)
GQSAKEAIEAALADSVKAYNSKDAAGVASKYMDDAAIFPLDMAPVDGRQNI
QKIAVQGINIDNIGVSGVKITTLDVQESGDFAFESGSFSIKGPGKDSKINDIAG
KYVEVWRKGQDGGWKLYRTIANLDPAK
(SEQ ID NO: 175)
GQSAKEAIEAALADFVKAYNSKDAAGVASKYMDDAAIFPPDNIAPVDGRQNI
QKILWQGLNIDNIGVSGPKLITINNIQESGDFAFESGSFSIKGPGTDSKINDIAG
KYVENAVRKGPDGGWKLYRTIANLDPAK
(SEQ ID NO: 176)
GQTAKEMEAALADEVICVYNSKDAAGVASKYMDDAAIFPLDNWADGRQNI
QKLWQGLNIDMGVSGVKLTTLDVQESGDFAFESGSFSLKGPGKDSKINDIAG
EYVENTWRKGQDGGWKLYRTIANLDPAK
(SEQ ID NO: 177)
GQSAKEAIEAALADFVKAYNSKDAAGVASKYMDDVAIFPLDMARVDGRQNI
QKIAVQGLMDMGVSEPKLTTLDVQESGDFAFESGSFSLKGPGKDSKINDIAGE
YVEVWRKGQDGGWRILYRTIANLDPAK
(SEQ ID NO: 178)
GQSAKEAIEAALADFVKAYNSKDAAGVASKYMDDAAIFPLDMARVDGRQNI
QKLWQGLMDMGVSEPKLTTLDVQESGDFAFESGSFSLKGPGKDSKLVDVAG
KYVEVWRKGQDGGWKLYRTIANLDPAK
(SEQ ID NO: 179)
GQSAKEAIEAALADFVKAYNSYJYITAGVASKYMDDAAIFPLDMAPVDGRQNI
QKLWQGLNIDMGVSGVKLTTLDVQESGDFAFESGSFSLKGPGKDSKINDIAG
KYVEVWRKGQDGGWKLYRTIANLDPAK
(SEQ ID NO: 180)
GQSAKEAIEAALADFVKVYNSKDAAGVASKYMDDAAIFPLDMAHVDGRQNI
QKIAVQGQMDMCWSGVKLITLDVQESGDFAFESGSFSLKGPGKDSKINDIAG
KYVEVWRKGQDGGWKLYRTIANLDPAK
(SEQ ID NO: 181)
GQSAKEAIEAALADFVKAYNSKDAAGVASKYMDDAAIFPLDMAPVDGRQNI
QKLWQGLMDMGVSGVKLTTLDVQESGDFASESGSFSIKGPGKDSKINDIAG
KYVEVWRKGQDGGWKLYRTIANLDPAK
(SEQ ID NO: 182)
GQSAKEAIEAALADFVKAYNSKDAAGVASKYMDDAAIFPPDMAPVDGRQN1
QKLWQGLMDMGVSGVKITTLDVQESGDFAFESGSFSIKGPGKDSKINDIAG
KYVEVWRKGQDGGWKLYRTIANLDPAK
(SEQ ID NO: 183)
DQSAKEAIEAALADFVKVYNSKDAAGVASKYMDDAVIFPLDMARVDGRQNI
QKLWQGLMDMGVSDPKFTTIJNVQESGDFAFESGSFSLKGPGKDSKLVDIAGI
YVEVWRKGQDGGLKLYRTIANWPAK
(SEQ ID NO: 184)
GQSAKEAIEAALADFVKAYNSKDAAGVASKYMDDAAIFPLDMAPVDGRQNI
QKLWQGLMDMGVSEPKFTTMNVQESGDFAFESGRFSLKGPGKDSKLVDIAG
KYVEVWRKGQGGGWKLYRTIANLDPAK
(SEQ ID NO: 185)
GQSAKEAIEAALADFVKVYNSKDAAGVASKYMDDAAIFPLDMAPVDGRQNI
QKLWQGLMDMGVSGVKITTINVQESGDFAFESGSFSIKGPGKDSKINDIAGI
YVEVWRKGQDGGWKLYRTIANLDPAK
(SEQ ID NO: 186)
GQSAKEAIEAALADFVKAYNSKDAAGVASKYMDDAAIFPLDMAPVDGRQNI
QKLWQGLMDMGVSEPKFTTLNVQESGDFAFESGSFSLKGPGKDSKLVDIAGI
YVEVWRKGQDGSWKLYRTIANLDPAN
(SEQ ID NO: 187)
GQSAKEAIEAALADFVKVYNSKDAAGVASKYMDDAVIFPMDMARVDGRQN
IQKLWQGLMDMGVSEPKFTTLNVQESGDFAFESGSFSLKGPGKDSKLVDIAGI
YVEVWRKGQDGGWKLYRTIANLDPAK
(SEQ ID NO: 188)
GQSAKVAIEAALADFVKVYNSKDVAGVASKYMDDAVIFPLDMARVDGRQNI
QKLWQGLMDMGVSEPKFITLNVQESGDFAFESGSFSLKGPGKDSKLVDIAGI
YVEVWRKGQDGGWKLYRTIANLDPAK
(SEQ ID NO: 189)
GQSAKVAIEAALADFVKVYNSKDVAGVASKYMDDAVIFPLDMARVDGRQNI
QKILAVQGLMDMGVSEPKFTILNVQESGDFAFESGIFSIKGPGKDSKRVDIAGI
YVEVWRKGQDGGWKLYRTIANLDPAK
(SEQ ID NO: 190)
GQSAKEAIEAALADFVKAYNGKDAAGVGSKYMDDAAIFPLDMARVDGRQNI
QKLWQGLMDTGVSEPKFTTLVVQESGDFAFESGSFSLKGPGPDSKLVDIAGK
YVEVWRKGQDGGWKLYRTIANLDPA
(SEQ ID NO: 191)
GQSAKEAIEAALADFVKAYNSKDAAGVASKYMDDAAIFPLDMAPVDGRQNI
QKLWQGLMDMGVSEPKFTTLNVQESGDFAFESGSFSLKGPSKDSKLVDIAGI
YVEVWRKGQDGGWKLYRTIANLDPAK
(SEQ ID NO: 192)
GHSAKEAIEAALADFVKAYNSKDAAGVASKYMDDAAIFPLDMAPVDGRQNI
QKLWQGLNIDLGVSEPKFTTLNVQESGDFAFESGGFSLKGPGKDSKINDIAGI
YVEVWRKGLDGCIWKLYRTIANLDPAK
(SEQ ID NO: 193)
GQSAKEAIEAVLADFVKAYNSKDAAGVASKYMDDAAIFPLDMAPVDGRQNI
QKIINVQGLMDMGVSEPKETTLNVQESGDFAFESGSFSLKGPGKDSKINDIAGI
YVEVWRKGQDGGWKLYRTIANLDPAK
(SEQ ID NO: 194)
DQSAKEAIEAALADFVKVYNSKNAAGVASKYMDDAVIFPLDMARVDGRQNI
QKILAVQGLMDMGVSEPKFITLNVQESGDFAFESGSFSLKGPGKDSKINDIAGI
YVEVWRKGQDGGWKLYRTIASLDPAK
(SEQ ID NO: 195)
GQSAKEAIEAALADFVKVYNSKDVAGVASKYMDDAVIFPLDMARVDGRQNI
QKILAVQGLMDMGVSEPKFITLNVQESGDFAFESGSFSLKGPGKDSKINDIAGI
YVEVWRKGQDGGWKLYRTIANLDPAK
(SEQ ID NO: 196)
GQSAKEAIEAALADFVKAYNSKDAAGVASKYMDDPAIFPLDMAPVDGRQNI
QKILAVQGLMDMGVSEPKFITLNVQESGDFAFESGSFSLKGPGKDSKINDIAGI
YVEVWRKGQDGGWKLYRTIANLDPAK
(SEQ ID NO: 197)
GQSAKEAIEAALADFVKVYNSKDAAGVASKYMDDAAIFPLDMAPVDGRQNI
QKIINVQGLMDMGVSEPKETTLNVQKSGDFAFESGSTSIKGPGKDSKINDIAGI
YVEVWRKGQDGGWKLYRTIANLDPAK
(SEQ ID NO: 198)
GQSGKEAIEAALADFVKAYNGKDAAGVASKYMDDAAIFPLDMARVDGRQNI
QKLWQGLNIDTGVSEPKFTTLVVQESGDFAFESGSFSLKGPGPDSRLVDIAGK
YVEVWRKGQDGGWKLYRTIANLDPAK
(SEQ ID NO: 199)
GQSAKEAIEAALADFVKAYNSKDAAGVANKYMDDAAIFPLDMAPVDGRQNI
QKLWQGLMDMGVSEPKLTTLDVQESGDFAFESGSFSLKGPGKDSKLVDIAG
KYVEVWRKGQDGGWKLYRTIANLDPAK
(SEQ ID NO: 200)
GQSAKEAIEAALADFVKAYNGKDAAGVASKYMDDAAIFPLDMARVDGRQNI
QKLWQGLNIDTGVSEPKFTTLVVQESGDFAFESGSFSLKGPGPDSKINDIAGK
YVEVWRKGQDGGWKLYRTIANLDPAK
(SEQ ID NO: 201)
GQSAKEAIEAALADFVKAYNSKDAAGVASKYMDDAAIFPLDMAPVDGRQNI
QKIINVQGLMDMGVSELKLITILDVQESGDFAFESGSFSIKGPGKDSKINDIAG
KYVEVWRKGQDGGWKLYRTIANLDPAK
(SEQ ID NO: 202)
GQSAKEAIEAALADFVKAYNSKDAAGVASKYMDDAAIFPLDMAPVDGRQNI
QKILAVQGLMDMGVSEPKYVILDVQESGDFAFESGSFSLKGPGKDSKINDIAG
KYVEVWRKGQDGGWKLYRTIANLDPAK
(SEQ ID NO: 203)
GQSAKEAIEAALADFVKAYNSKDAAGVASKYMDDAAIFPLDMAPVDGRQNI
IKLWQGIAIDLGNISGPKFITINVQESGDFAFESGSFSLKGPGKDSKINDIAGK
YVEVWRKGQDGGWKLYRTIANLDTAK
(SEQ ID NO: 204)
GQSAKEAIEAALADFVKAYNSKDVAGVASKYMDDAVIFPLDMAPVDGRQNI
QMINVQGIAIDNICVSGVKITTLDVQESGDFAFESGSFSLKGPGKDSKINDIAG
KYVFNWRKGQDGGWKLYRTIANLDPAK
(SEQ ID NO: 205)
GQSAKGAIEAALADFVKAYNSKDAAGVASKYMDDAAIFPLDNIAPVDGRQNI
QKLWLGLMDMGVSEPKLTTLDVQESGDFAFESGSFSLKGPGKDSKLVDIAGK
YVEVWRKGQDGGWKLYRTIANLDPAK
(SEQ ID NO: 206)
GQSAKEAIEAALADFVKAYNSKDAAGVASKYMDDAAIFPLDMARVDGRRNI
QKLWQGLMDNIGVSEPKLTTLDVQESGDFAFESGSFSLKGPGKDSKLVDIAG
KYVEVWRKGQDGGWKLYRTIANLDPAK
(SEQ ID NO: 207)
GQSSKEALEVALADFVKAYNSKDAAGVASKYNIDDAAIFPLDMARVDGRQN1
WINVQGLMDNIGVSEPKLTTLDVQESGDFAFESGSFSLKGPSKDSKINDIAGK
YVEVWRKGPDGGWRILYRTIANLDPAK
(SEQ ID NO: 208)
GQSAKEAIEAALADFVKAYNSKDAAGVASKYMDDAAIFPLDMAPVDGRQNI
QELWQGLMDMGVSELKLTTLDVQESGDFAFESGNFSLKGPGKDSKINDIAG
KYVFNWRKGQDGGWKLYRTIANLDPAK
(SEQ ID NO: 209)
GQSAKEAIEAALADFVKAYNSKDAAGVASKYMDDAAIFPLDMARVDGRQNI
WINVQGLMDNIGVSEPKLTTLDVQESGDFAFESGSFCLKGPGKDSKINDIAG
KYVFNWRKGQDGGWKLYRTIANLDPAK
(SEQ ID NO: 210)
GQSAKEAIEAALADFVKAYNSKDAAGVASKYMDDAAIFPLEMAPVDGRQNI
QKLWQGLMDNIGVSEPKLTTLDVQESGDFAFESGSFSLKGPGKDSKLVDIAG
KYVEVWRKGQDGGWKLYRTIANLDPAK
(SEQ ID NO: 211)
GQSAKEAIEAALADFVKAYNSKDAAGVASKYMDDATIFPLDMARVDGRQNI
QKLWQGLMDNIGVSELKSTTLDVQESGDFAFESGSFSLKGPGKDSKLVDVAG
KYVVIAVRKGQDGGWKLYRTISNLDPAK
(SEQ ID NO: 212)
GQSAKEAIEAALADFVKAYNSKDAAGVASKYMDDAAIFPLDMARVDGRQNI
QKILAVQGLMDNIGVSELKSTTLDVQESGDFAFESGSFSLRGPGKDSKINDVAG
KYVVIAVRKGQDGGWKLYRTISNLDPAK
(SEQ ID NO: 213)
GQSAKEAIEAALADFVKAYNSKDAAGVASKYMDDAAIFPLDMARVDGRQNI
WINVQGLMDNIGVSEPKLTTLDVQESGDFAFESGSFSLKGPGKDIKINDIAGK
YVEVWRKGQDGGWKLYRTIANLDPAK
(SEQ ID NO: 214)
GQSAKEAIEAALADEVKAYNSKDAAGVASKYNIDDAAIFPLDMARVDGRQNI
QKIAVQGLNIDMGVSEPKLTTLGVQESGDENFESCISFSLKGPGKDNKINDIAG
KYVEVWRKGQDGGWKLYRTIANLDPAK
(SEQ ID NO: 215)
GQSAKEAlEAALADEVKAYNSKDAAGVASKYNIDDAAIFPLDMARNIDGRQNI
QKIAVQGLMDMGVSEPKETTLNVQESGDFAFESGSFSLKCIPGKDSKINDIAGI
YVEVWRKGQDGGWKLYRTIANLDPAK
(SEQ ID NO: 216)
GQSAKVAIFAALADFVKVYNSKDAAGVASKYMDDAAIFPLDNIARVDGRQNI
QKIAVQGLMDMGVSEPKETTLNVQESGDFAFESGSFSLKCIPGKDSKINDIAG
NYVENWRKGQGGGWKLYRTIANLDPAK
(SEQ ID NO: 217)
GQSAKEAIEAALADFVKAYNSKDAAGVASKYMDDAAIFPLDMAPVDGRQNI
QKLWQGLNIDMGVSGVKLTTLDVQESGDFAFESGSFSLKGPGKDSKINDIAG
KYVEVWRKGQDGGWKLYRTIANLDPAN
(SEQ ID NO: 218)
GQSAKVAIEAALADFVKVYNSKDAAGVASKYMDDAAIFPLDNIAPVDGRQNI
QKLWQGLMDMGVSEPKFITLNVQESGDFAFESGSFSLKCIPGKDSKINDIAG
NYVENWRKGQGGGWKLYRTIANLDPAK
(SEQ ID NO: 219)
GQSAKEAIEAALADEVKAYNSKDAAGVASKYNIDDAAIFPLDMARVDGRQNI
QKLWQGLMDMGVSEPKFITLNVQESGDFAFESGSFSLKCIPGKDSKINDIAG
NYVENWRKGQDGGWKLYRTIANLDPAK
(SEQ ID NO: 220)
GQSAKEAIEAALADEVKAYNSKDAAGVASKYNIDDAAIFPLDMARVDGRQNI
QKLWQGLMDMGVSELKETTLDVQESCIDEAFESGSFSIKGPCIKDSKINDIAG
KYVENAVRKADPPPSSEGTREMVPYN
(SEQ ID NO: 221)
GQSAKEAIEAALADEVKAYNSKDAAGVASKYNIDDAAIFPLDMARVDGRQNI
QKLWQGLMDMGVSELKLTTLDVRESGDENFESGSFSLKGPGKDSKLVDIAG
KYVEVWRKGQDGGWKLYRTISNLDPAK
(SEQ ID NO: 222)
GQSAKEAIEAALADEVKAYNSKDAAGVASKYNIDDAAIFPLDMARVDGRQNI
QKLWQGLMDMGVSELKETTLDVQESCIDEAFESGSFSIKGPCIKDSKINDVAG
KYVEVWRKGQDGGWKLYRTIANLDPAK
(SEQ ID NO: 223)
GQSAKEAlEAALADEVKAYNSKDAAGVASKYNIDDAAIFPLDMARVDGRQNI
QKLWQGLMDMGVSELKLITLDVQESCIDEAFESGSFSIKGPCIKDSKINDVAG
KYVEVWRKGQDGGWKLYRTISNLDPAK
(SEQ ID NO: 224)
GQSAKEAIEAALADFVKAYNSKDAAGVASKYMDDAAIFPLDMARVDGRQN
TQKLWQGLMDMGVSELKLTTLDVQESGDFAFESGSFSLKGPGKDSKLVDIAG
KYVEVWRKGQDGGWKLYRVIVNLDPAK
(SEQ ID NO: 225)
GQSAKGAIEAALADFVKAYNSKDAAGVASKYMDDAAIFPLDMARVDGRQNI
QKIAVQGLMDMGVSELKLITLDVQESCIDFAFESGSFSIKGPCIKDSKINDIAG
KYVENWRKGQDGGWKLYRVIVNLDPAK
(SEQ ID NO: 226)
GQSAKEAIEAALADFVKAYNSKDAAGVASKYMDDAAIFPLDMARVDGRQNI
QKLWQGLMDMGVSELKLITLDVQESCIDFAFESGSFSIKGPCIKDSKINDIAG
KYVENWRKGQDGGWKLYRVIVNLDPAK
(SEQ ID NO: 227)
GQSAKEAIEAALADFVKAYNSKDAAGVASKYMDDAAIFPLDMARVDGRQNI
QKLWQGLMDMGVSELKLITLDVQESCIDFAFESGSFSIKGPCIKDSKINDIAG
KYVENWRKGQDGGWKLYRAIANLDPAK
(SEQ ID NO: 228)
ARSAKEAIEAALADFVKAYNSKDAAGVASKYMDDAAIFPLDMARVDGIQNI
QKLWQGLMDMGVSELKLITLDVQESCIDFAFESGSFSIKGPCIKDSKINDIAG
KYVENWRKGQDGGWKLYRAIANLDPAK
(SEQ ID NO: 229)
GQSAKEAIEAALADFVKAYNSKDAAGVASKYMDDAAIFPLDMARVDGRQNI
QKIAVQGLMDMGVSENTKLTTLDVQGSGDFAFESGSFSAKGPGKDSKINDMA
GKYVEVWRKGQDGGWKLYRVIANLDPAK
(SEQ ID NO: 230)
GQSAKEAIEAALADFVKAYNSKDAAGVACKYMDDAAIFPLDMARVDGRQNI
QKIAVQGLMDMGVSENTKLTTLDVQESGDFAFESCISFSAKCIPGKDSKINDMA
GKYVEVWRKGQDGGWKLYRVIANLDPAK

In a further embodiment, the polypeptides of any embodiment of any aspect of the invention may further comprise a tag, such as a detectable moiety. The tag(s) can be linked to the polypeptide through covalent bonding, including, but not limited to, disulfide bonding, hydrogen bonding, electrostatic bonding, nucleophilc (i.e. Cys, Lys) conjugation chemistry, recombinant fusion and conformational bonding. Alternatively, the tag(s) can be linked to the polypeptide by means of one or more linking compounds. Techniques for conjugating tags to polypeptides are well known to the skilled artisan. Polypeptides comprising a detectable tag can be used diagnostically to, for example, identify the presence of vitamin D3 or one of its metabolites or other steroid in a sample of interest. However, they may also be used for other detection and/or analytical and/or diagnostic purposes. Any suitable detection tag can be used, including but not limited to enzymes, prosthetic groups, fluorescent materials, luminescent materials, bioluminescent materials, radioactive materials, positron emitting metals, and nonradioactive paramagnetic metal ions. The tag used will depend on the specific detection/analysis/diagnosis techniques and/or methods used such as immunohistochemical staining of (tissue) samples, flow cytometric detection, scanning laser cytometric detection, fluorescent immunoassays, enzyme-linked immunosorbent assays (ELISAs), radioimmunoassays (RIAs), bioassays (e.g., neutralization assays), Western blotting applications, etc. For immunohistochemical staining of tissue samples preferred tags are enzymes that catalyze production and local deposition of a detectable product. Enzymes typically conjugated to polypeptides to permit their immunohistochemical visualization are well known and include, but are not limited to, acetylcholinesterase, alkaline phosphatase, beta-galactosidase, glucose oxidase, horseradish peroxidase, and urease. Typical substrates for production and deposition of visually detectable products are also well known to the skilled person in the art. The polypeptides can be labeled using colloidal gold or they can be labeled with radioisotopes, such as 33P, 32P, 35S, 3H, and 125I. Polypeptides of the invention can be attached to radionuclides directly or indirectly via a chelating agent by methods well known in the art.

When the polypeptides of the invention are used for flow cytometric detections, scanning laser cytometric detections, or fluorescent immunoassays, the tag may comprise, for example, a fluorophore. A wide variety of fluorophores useful for fluorescently labeling the polypeptides of the invention are known to the skilled artisan. When the polypeptides are used for in vivo diagnostic use, the tag can comprise, for example, magnetic resonance imaging (MRI) contrast agents, such as gadolinium diethylenetriaminepentaacetic acid, to ultrasound contrast agents or to X-ray contrast agents, or by radioisotopic labeling.

The polypeptides of the invention can also be attached to solid supports, which are particularly useful for in vitro assays or purification of vitamin D3 or one of its metabolites. Such solid supports might be porous or nonporous, planar or nonplanar and include, but are not limited to, glass, cellulose, polyacrylamide, nylon, polystyrene, polyvinyl chloride or polypropylene supports. The polypeptides can also, for example, usefully be conjugated to filtration media, such as NHS-activated Sepharose or CNBr-activated Sepharose for purposes of affinity chromatography. They can also usefully be attached to paramagnetic microspheres, typically by biotin-streptavidin interaction. As another example, the polypeptides of the invention can usefully be attached to the surface of a microtiter plate for ELISA.

In a further aspect, the present invention provides isolated nucleic acids encoding a polypeptide of the present invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present invention provides recombinant expression vectors comprising the isolated nucleic acid of any aspect of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989, Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

In a still further aspect, the present invention provides host cells that have been transfected with the recombinant expression vectors disclosed herein, wherein the host cells can be either prokaryotic (such as bacteria) or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney. 1987. Liss, Inc. New York, N.Y.). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

In another aspect, the invention provides methods for detecting vitamin D3 or one of its metabolites, such as 25-D3, comprising contacting a sample of interest with a detectable polypeptide of the invention under suitable conditions for binding the detectable polypeptide to vitamin D3 or one of its metabolites (such as 25-D3) present in the sample to form a polypeptide—vitamin D3 (or, for example, a polypeptide-25-D3)) binding complex, and detecting the binding complex. In one embodiment, the sample is a biological sample, including but not limited to blood, serum, nasal secretions, tissue or other biological material from a subject to be tested. The polypeptides of the invention for use in this aspect may comprise a conjugate as disclosed above, to provide a tag useful for any detection technique suitable for a given assay. The tag used will depend on the specific detection/analysis/diagnosis techniques and/or methods used. The methods may be carried out in solution, or the polypeptide(s) of the invention may be bound or attached to a carrier or substrate, e.g., microtiter plates (ex: for ELISA), membranes and beads, etc. Carriers or substrates may be made of glass, plastic (e.g., polystyrene), polysaccharides, nylon, nitrocellulose, or teflon, etc. The surface of such supports may be solid or porous and of any convenient shape.

In one embodiment, the polypeptide is a polypeptide according to SEQ ID NOS:2-3, or SEQ ID NOS: 4-230, each of which include the V107E modification relative to CRL2, which is shown in the examples that follow to significantly increase specificity for 25-D3 relative to D3. In specific embodiments, the polypeptide comprises or consists of SEQ ID NOS: 29 or 30 (CDL2.1 or CDL2.2).

In various non-limiting embodiments, the methods can be used for diagnosis, prognosis, and/or treatment monitoring of autoimmune or chronic diseases including but not limited to multiple sclerosis, systemic lupus erythematosus, and fibromyalgia.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

### EXAMPLES

### Abstract

While previous efforts in designing proteins to bind small molecules have yielded some successes with hydrophilic targets, binding hydrophobic molecules is a qualitatively different challenge. Having few hydrogen bonds and a primarily hydrophobic surface makes it incredibly difficult to design binders for specificity over chemically similar molecules.

We developed a computational protocol that first performs an iterative search and vastly increases sampling when compared with previous protocols. This results in a tailored method for designing highly shape complementarity designs. We demonstrated the quality of these design by targeting the ligand 25-hydroxycholecaliferol (25-D3). 25-D3 is the hormonally active form of vitamin D3, is a common target for medical diagnostics, and would benefit from a greater distinction between 25-D3 and chemically similar metabolites such as vitamin D3 and vitamin D2.

Initial designed binders for 25-D3 showed negligible specificity over the chemically similar target vitamin D3. After directed evolution, these designs became more specific for their intended ligand, and resulted in nanomolar binders for 25-D3. Mutations suggest this specificity improvement is due to a change in backbone structure or protein stability as opposed to changes to the designed hydrogen bonding residues. A crystal structure was solved for a 25-D3 binder. Our design protocol has demonstrated the ability to create specific binding proteins for the hydrophobic ligand 25-D3.

Results

Computational Protocol for Design of Small Hydrophobic Molecules

The strategy to design a computational protocol to generate protein binders for hydrophobic small molecules focuses on high shape complementarity between the small molecule and the protein Initially, the small molecule of interest is placed into protein pockets with high shape complementarity and sampling is expanded by including crystal structures of the top scoring topologies. Due to experimental restrictions with labeling of the ligands, the orientation of linker is used as a filter to remove placements where the linker points into the protein and not out. Next, the ligand interaction is systematically sampled by generating spatial perturbations of its initial placement, in order to increase its shape complementarity between the protein and ligand. Optimization of small physicochemical interactions in this way can result in discrete amino acid identity changes and improves sampling of the sometimes jagged energy landscape. The interactions between the ligand and protein are optimized using the ROSETTA ENERGY® function and the potential designs are filtered e.g. on shape complementarity. Lastly, the computational designs are manually inspected and rational substitutions are tested using ROSETTA®. The computational protocol was tested on the hydrophobic ligand 25-hydroxycholecaliferol (25-D3).

### 25-D3 Designs

From the computational protocol, 28 designs were ordered in 6 different scaffold classes targeting the ligand 25-D3. 7 out of 28 designs showed a signal via yeast display and flow cytometry that indicates successful binding. Of these designs, the tightest, named CDL1, has a NTF2 topology (PDB ID: 1Z1S) which are known to bind steroids—interestingly the native sequence did not show any binding for 25-D3 so it was necessary to introduce mutations to repurpose its function. To increase the binding affinity the initial computational design, CDL1, was evolved via error prone mutagenesis (ep-PCR) into a variant CDL1.1, which contains additional mutations P46S, R55A, H68P, and G136V The P46S and H68P mutations are located near the entrance of the binding site where P46S makes a loop more flexible while H68P rigidifies a loop. The two other mutations are distal to the binding pocket and seem to increase stability of the scaffold by e.g. increasing helix-helix packing (R55A). From yeast surface display, the initial design has a Kd of approximately 2 uM where the evolved variant has an improved affinity with an estimated Kd of 229 nM In a sensor application, specificity against the non-hydroxylated vitamin D3 would be an important distinction. The design CDL1 did not show a significant preference for D3-25 over D3, however, the evolved variant CDL1.1 increased its specificity to about two fold over CDL1. (see Table 4).

CDL1 = 6234
(SEQ ID NO: 231)
SGREQGHMNAKEILVHALRLVENGDARGFCDLFHPEGVMEFPYAPPGYK
TRFEGRETIWAHMRLFPEHLTIRFTDVQFYETADPDLAIGEFHGDGVAT
VSGGKLAWDFISVLRTRDGQILLSRIFWNPLRHLEALGGVEAAAKIVQG
A
CDL1.1 = N3X-AD4 (Truncated as well as mutated
from 6234)
(SEQ ID NO: 232)
NLYFQGHMNAKEILVHALRLVENGDARGFCDLFHPEGVMEFPYAPSGYK
TRFEGAETIWAHMRLFPEPLTIRFTDVQFYETADPDLAIGEFHGDGVAT
VSGGKLAQDFISVLRTRDGQILLSRIFWNPLRHLEALV

We discovered another binder for 25-D3, referred to here as CDL2. This binder showed an exceptionally strong signal when expressed on yeast and tested for a binding signal against a biotinylated 25-D3 molecule via flow cytometry. It was further evolved to investigate and improve its specificity and affinity. To test a broader number of mutants, CDL2 was optimized using ep-PCR as well as small computationally guided library. The computationally guided library was constructed by docking the ligand into the binding site and optimizing the interactions between 25-D3 and the protein using ROSETTA®. To increase the sampling of the ligand, short MD simulations were performed to make small perturbations of the backbone. These computational variants, as well as variants generated via error prone mutagenesis, were expressed on yeast and sorted via fluorescence activated cell sorting. Individual designs sequenced from various rounds of mutagenesis and sorting were sequenced during the evolution to inform further design and mutagenesis strategies. One evolved variant, CDL2.1, incorporated 10 mutations scattered around the protein. Another evolved variant CDL2.2 is the most advanced variant from the directed evolution efforts.

TABLE 4

| Desig- | | | | Approximate |
|---|---|---|---|---|
| nation | PDB ID | Protein Fold | Ligand Target | Kd |
| CDL1 | 1Z1S | Putative | 25- | 1300 nM |
| | | Isomerase | hydroxycholecalciferol | |
| CDL1.1 | 1Z1S | Putative | 25- | 229 nM |
| | | Isomerase | hydroxycholecalciferol | |
| CDL2 | 3HX8 | Ketosteroid | 25- | 2100 nM |
| | | Isomerase | hydroxycholecalciferol | |
| CDL2.1 | 3HX8 | Ketosteroid | 25- | 319 nM |
| | | Isomerase | hydroxycholecalciferol | |

Next, crystal structures of an evolved variant of CDL2, referred to as CDL2.1, were solved where the ligand was within 1.066° Armsd of the docked placement of the ligand.

The design strategy fir binders targeting 25-D3 or any hydrophobic small molecule is to favor s highly shape complementary pocket with tight packing, as adequate specificity through hydrogen bonds is sometimes not possible. Hydrogen bonding interactions are not treated as a strict requirement in initial design rounds. During iterative refinement involving repeated rounds of ligand perturbation and ROSETTA® design, a selection pressure for hydrogen bonds is applied. The primary difference between the molecule 25-D3 over the similar molecule, vitamin D3, is a tertiary hydroxyl group, and is the primary design target to introduce specificity between the two molecules. 25-D3 binding design CDL1 is based on the scaffold with PDB ID 1Z1S, a putative isomerase with unknown function. CDL1 contains 8 mutations from 1Z1S, which primarily replace the native binding pocket with shape complementary hydrophobic residues. CDL1 accomplishes the recognition of the tertiary hydroxyl via the design of two serine residues deep in the binding pocket.

CDL2 is based on the scaffold with PDB ID 3HX8, a putative ketosteroid isomerase. CDL2 was evolved against 25-D3 for a potential use as a diagnostic. Several crystal structures were solved of evolved variants, the tightest of which is named CDL2.1. The crystal structure of CDL2.1 contains a significant backbone movement near a key residue, 106E. This mutation was found through directed evolution and, once found, provided the majority of the specificity for 25-D3 over vitamin D3 and significantly increased affinity. We therefore consider it an important interaction to be able to correctly model to improve future design efforts.

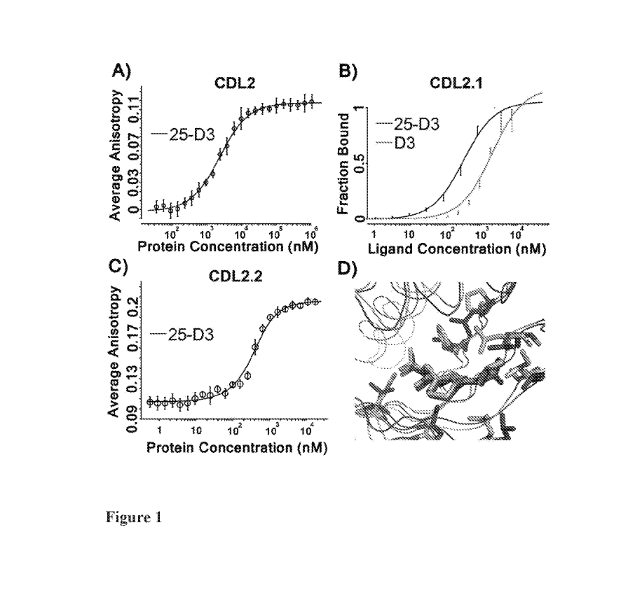

We used ROSETTA DOCK® to probe the quality of the designs to bind 25-D3. When 25-D3 is docked into the crystal structure, the ligand position agrees within 0.068° A of the crystal ligand position and additionally shows a favorable docking profile, where the ligand interface energy decreases as RMSD of the docked ligand approaches that of the ligand in the crystal structure. See FIG. 1D.

### Materials and Methods

### Selection of Protein Structures

The scaffolds used were crystal structures from the Protein Data Bank (PDB) [9] from 2013. Filters were applied to ensure the protein sizes were no larger than 350 amino acids, contained heteroatoms, and had a resolution 2,5° A or better. Crystal structures were also collected from the binding mother of all databases (MOAD) [10] from 2010, as well as homologous proteins shown to have expressed well or have had success being computationally designed in the past.

### Ligand Conformer Generation and Placement

Conformers for the target ligands were generated using OPENBABEL® [11]. The PATCHDOCK® [12] algorithm was used to place the lowest energy ligand conformer into a protein pocket with high shape complementarity. We filtered these Patch-dock outputs based on the ligand's orientation and solvent accessibility. To increase sampling of scaffold backbones and binding pocket shapes, the surviving design models were expanded to include scaffolds in the same pfam [13] and a variety of sequence variants were generated. PATCHDOCK®-based placement was again applied to each one of these scaffold variants with an additional 20 to 40 low energy ligand conformers.

### Design of Proteins

Docked poses were again filtered, as described above, before being expanded by making translational and rotational perturbations to the ligand positions. Each one of these perturbed models underwent further design to optimize the sequence for minimal predicted interaction energy between the ligand and protein. Models were filtered using the Rosetta interface energy and shape complementarity. These surviving models again underwent perturbation, ROSETTA DESIGN®, and filtering in an iterative process. In successive rounds, the amplitude of perturbation was decreased, density of sampling was increased, and score filters were made stricter. Designs were manually inspected e.g., to revert substitutions distal to the binding site back to native identity. The final designs were ordered for experimental testing

### Experimental Verification of Design Using Yeast

Binding activity yeast surface display and flow cytometry, according to methods previously described by Wittrup et al. [14]

### MD Simulations

Short MD simulations were set up for design CDT-2. The coordinates were prepared using AMBERTOOLS® 14 with the ff14SB force field. The starting coordi-nates were minimized for 20,000 steps with 10,000 steepest descent (SD) followed by 10,000 conjugated gradient (GC). The minimized structures were solvated and neutralized by adding counter ions to the system. The solvent was minimized by restraining residue 1 to 128 using a three of 500.0 kcal/mol/A. SD for 10.1 steps followed by 10,000 steps of GC. The whole complex was minimized using 10,000 steps of SD followed by 10,000 GC. The system was heated to 300 K applying a restraint of 50.0 kcal/mol/°Aon residue 1 to 128 for 50,000 steps using an integration step of 2 fs. 50 trajectories with different initial velocities were produced keeping the temperature at 300 K by using a Langevin thermo-stat with a collision frequency of 2 ps⁻¹ integrated using a step of 2 fs keeping the pressure at 1 atm using a barostat. Coordinates were saved every 10 ps.

### REFERENCES

[1] Design of a novel globular protein fold with atomic-level accuracy, Science 302 (5649) (2003) 1364-1368.
[2] Computational de novo design of a self-assembling peptide with predefined structure, Journal of Molecular Biology 427 (2) (2015) 550-562.
[3] Kemp elimination catalysts by computational enzyme design, Nature 453 (2008) 190-195.
[4] De novo computational design of retro-aldol enzymes, Science 319 (5868) (2008) 1387-1391.
[5] Computational design of proteins targeting the conserved stem region of influenza hemagglutinin, Science 332 (6031) (2011) 816-821.
[6] Exploitation of binding energy for catalysis and design, Nature 461 (2009) 1300-1304.
[7] Computational redesign of endonuclease DNA binding and cleavage specificity, Nature 441 (2006) 656-659.
[8] Computational design of ligand-binding proteins with high affinity and selectivity, Nature 501 (2013)212-216.
[9] The protein data bank, Nucleic Acids Research 28 (4) (2000) 235-242.
[10] Binding moad (mother of all databases)., Proteins 60 (2005)333-40.
[11] Open babel: An open chemical toolbox, Journal of Cheminformatics 3 (33).
[12] Patchdock and symmdock: servers for rigid and symmetric docking, Nucleic Acids Res 33 (Web Server issue) (2005) W363-7.
[13] The pfam protein families database., Nucleic Acids Res 38 (2010) D211-22.
[14] Isolating and engineering human antibodies using yeast surface display, Nat Protoc 1 (2) (2007) 755-68.

1. An isolated polypeptide comprising a polypeptide at least 70% identical over the full length of the amino acid sequence of SEQ ID NO:1.

2. The isolated polypeptide of claim 1, comprising a polypeptide at least 80% identical over the full length of the amino acid sequence of SEQ ID NO:1.

3. The isolated polypeptide of claim 1, comprising a polypeptide at least 90% identical over the full length of the amino acid sequence of SEQ ID NO:1.

4. The isolated polypeptide of claim 1, comprising the amino acid sequence of SEQ ID NO:2.

5. The isolated polypeptide of claim 1, comprising the amino acid sequence of SEQ ID NO:3.

6. The isolated polypeptide of claim 1, comprising the amino acid sequence of a peptide selected from the group consisting of SEQ ID NOS: 1-230.

7. The isolated polypeptide of claim 1, comprising the amino acid sequence of a peptide selected from the group consisting of SEQ ID NOS: 29-30.

8. An isolated polypeptide comprising the amino acid sequence of SEQ ID NO: 231 or 232.

9. The isolated polypeptide of claim 1, further comprising a detectable tag.

10. An isolated nucleic acid encoding the polypeptide of claim 1.

11. A recombinant expression vector comprising the isolated nucleic acid of claim 10 operably linked to a control sequence.

12. A recombinant host cell comprising the recombinant expression vector of claim 11.

13. A method for detecting vitamin D3 or one of its metabolites, comprising:
(a) contacting a sample of interest with a polypeptide according to claim 1 under suitable conditions for binding the polypeptide to vitamin D3 or one of its metabolites present in the sample to form a polypeptide-vitamin D3 (or one of its metabolites) binding complex, and
(b) detecting the binding complex.

14. The method of claim 13, wherein the binding complex comprises a polypeptide-25-D3 binding complex.

15. The method of claim 13, wherein the polypeptide is selected from the group consisting of SEQ ID NOS: 29-30.

16. The method of claim 13, wherein the method is used for diagnosis, prognosis, and/or treatment monitoring of autoimmune or chronic diseases including but not limited to multiple sclerosis, systemic lupus erythematosus, and fibromyalgia.
